(19) United States
(12) Patent Application Publication
(10) Pub. No.: US 2022/0220489 A1
(43) Pub. Date: Jul. 14, 2022

(54) INDUCIBLE PLASMID-SELF-DESTRUCTION ASSITED RECOMBINATION

(71) Applicant: Deerland Probiotics & Enzymes A/S, Hundested (DK)

(72) Inventors: Fanglei Zuo, Huddinge (SE); Zhu Zeng, Huddinge (SE); Harold Marcotte, Huddinge (SE)

(21) Appl. No.: 17/595,273

(22) PCT Filed: May 15, 2020
(86) PCT No.: PCT/EP2020/063668

§ 371 Date: Nov. 12, 2021

(30) Foreign Application Priority Data
May 15, 2019 (EP) 19174715.3

(51) Int. Cl. C12N 15/74; C07K 14/335; C12N 15/10

(52) U.S. Cl. CPC C12N 15/746; C07K 14/335; C12N 2810/55; C12N 2800/30; C12N 15/102

(57) ABSTRACT

The present invention provides a circular DNA vector, which may be used to introduce specific a mutation in a target region of a host cell. The present invention further provides methods using the circular DNA vector for generating engineered host cells. The circular DNA vector and methods are useful for studying gene functions and generating cells producing recombinant gene products.

### FIELD OF THE INVENTION

The present invention provides a circular DNA vector, which may be used to introduce specific a mutation in a target region of a host cell. The present invention further provides methods for using the circular DNA vector for generating engineered host cells. The circular DNA vector and methods are useful for studying gene functions and generating cells producing recombinant gene products.

### BACKGROUND OF THE INVENTION

Lactobacilli has been extensively used as probiotics and have become increasingly studied as delivery vehicles of medically relevant recombinant proteins to mucosal surfaces. In contrast, the genetic tools (especially mutagenesis) to investigate and enhanced their probiotic activity are rather poorly developed. The traditional genome engineering methods are largely dependent on the bacterial transformation efficiency. This defect can be overcome by the conditional replicate plasmid assisted recombineering, such as plasmids containing a thermosensitive replication origin of pWV01, but the latter may have a limited host range. Therefore, more flexible and effective genome editing strategies need to be developed for a better understanding and application of these health-promoting microorganisms.

### SUMMARY OF THE INVENTION

A first aspect of the present invention provides a circular DNA vector comprising:
(a) a selectable marker gene sequence, wherein said marker gene sequence is operably linked to a first promoter sequence
(b) a multiple cloning site, wherein said multiple cloning site optionally comprises a gene targeting sequence,
(c) a sequence encoding a site-specific recombinase, wherein said sequence is operably linked to a second promoter sequence, wherein said second promoter is inducible,
(d) a replicon sequence,
(e) two target sites for said site-specific recombinase,
   wherein vector comprises a first region flanked on each side by one of said target sites for said site-specific recombinase and wherein said region comprises (a) and (b) with the proviso that (c) and (d) are not within said first region.

The circular DNA vector is useful as targeting vector for targeted integration of a (gene) sequence in the genome host by homologous recombination. The circular DNA vector is particularly useful for gene targeting in host cells, which are generally known to be difficult to transform, such as species of Lactobacilli and Bifidobacteria.

The circular DNA vector is particularly useful for gene targeting in accordance with the methods of the present invention.

Accordingly, in a second aspect of the present invention provides a method for introducing recombination between a circular DNA vector and a target region of the genome of a host cell, said method comprising the steps of:
(i) introducing a circular DNA vector comprising a gene targeting sequence according to the present invention in a host cell, wherein said target sequence comprising flanking sequences comprising at least about 200 consecutive nucleosides having a sequence identity of at least 80% to the corresponding region of the target region of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and
(iv) selecting a host cell, wherein (a) have been excised from the genome of the host cell obtained under (iii) by a second homologous recombination event between the flanking sequences of the targeting sequence and the target region of the genome.

In a third aspect, the present invention provides a method for generating a host cell having a mutation in a target gene of the genome of a host cell, said method comprising the steps of:
(i) introducing a circular DNA vector comprising a gene targeting sequence according to the present invention in a host cell, wherein said target sequence comprising flanking sequences comprising at least about 200 consecutive nucleosides having a sequence identity of at least 80% to the corresponding region of the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and (iv) selecting a host cell, wherein (a) have been excised from the genome of the host cell obtained under (iii) by a second homologous recombination event between the flanking sequences of the targeting sequence and the target region of the genome and, wherein said host cell comprises a mutation in said target region.

In a fourth aspect, the invention provides a method for generating a host cell having loss of function in a target gene of the genome of a host cell, said method comprising the steps of:
(i) introducing a circular DNA vector comprising a gene targeting sequence according to the present invention in a host cell, wherein said target sequence comprising flanking sequences comprising at least about 200 consecutive nucleosides having a sequence identity of at least 80% to the corresponding region of the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and (iv) selecting a host cell, wherein (a) have been excised from the genome of the host cell obtained under (iii) by a second homologous recombination event between the flanking sequences of the targeting sequence and the target region of the genome and, wherein said host cell comprises a loss of function of said target gene.

In a fifth aspect, the invention provides a method for generating a host cell having gain of function in a target gene of the genome of a host cell, said method comprising the steps of:
(i) introducing a circular DNA vector comprising a gene targeting sequence according to the present invention in a host cell, wherein said target sequence comprising flanking sequences comprising at least about 200 consecutive nucleosides having a sequence identity of at least 80% to the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and (iv) selecting a host cell, wherein (a) have been excised from the genome of the host cell obtained under (iii) by a second homologous recombination event between the flanking sequences of the targeting sequence and the target region of the genome and, wherein said host cell comprises a gain of function of said target gene.

In a sixth aspect, the invention provides a method for preparing a host cell expressing a recombinant polypeptide, said method comprising the steps of:
(i) introducing a circular DNA vector comprising a gene targeting sequence according to the present invention in a host cell, wherein said target sequence comprising flanking sequences comprising at least about 200 consecutive nucleosides having a sequence identity of at least 80% to the corresponding region of the target region of the host cell genome, and wherein said gene targeting sequence encodes a recombinant polypeptide,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and
(iv) selecting a host cell, wherein (a) have been excised from the genome of the host cell obtained under (iii) by a second homologous recombination event between the flanking sequences of the targeting sequence and the target region of the genome.

A further aspect of the present invention provides a recombinant host cell obtained by any of the methods of the present invention.

In yet a further aspect, the present invention provides the use of the circular vector of the present invention for introducing a gene sequence in the genome of a host cell.

In one further aspect, present invention provides the use of the circular vector of the present invention for increasing tissue adhesion of a host cell.

### BRIEF DESCRIPTION OF THE DRAWINGS

FIG. 1 IPSD strategy for bacterial recombineering. a, Schematic illustration of inducible plasmid self-destruction. A vector in which the replicon and the antibiotic resistance gene are separated by two oriented six fragments was constructed. Upon addition of the inducer and β mediated recombination, the vector loses function due to the excision of the replicon. Rep, replicon; Pro, controlled expression promoter; Rec, recombinase; six: two oriented six target sequence sites; Ar, antibiotic resistance gene; MCS, multiple cloning sites. b, Schematic illustration of IPSD assisted bacterial recombineering, including gene deletion, insertion, and replacement (indicated by an asterisk). After recombination, the ratio of colonies harboring the episomal vector decreased (blue arrows) while the ratio of colonies with integrated DNA fragment through singlecrossover increased (red arrows). The singlecrossover mutant colonies could be screened by PCR and the doublecrossover clones could be selected by counterselection. The homologous ends flanking the target gene are indicated by A and B. The target gene on the chromosome is represented by a pink rectangle. c, Growth of the indicated L. gasseri DSM 14869 strain on MRS agar plate supplemented with 10 μg/ml chloramphenicol and in presence or not of 100 ng/ml SpplP.

FIG. 2

The physical map of IPSD vector pINTZrec. Cmr, chloramphenicol resistance gene; Rec, recombinase gene β.

FIG. 3 IPSD assisted gene deletion and insertion in lactobacilli. a,b, The L. gasseri DSM 14869 upp gene was deleted and the deletion mutant verified by PCR using primer pairs uppleft-F and uppright-R. c, The L. gasseri DSM 14869 upp mutant showed resistance to 100 μg/ml of 5-FU compared to the WT.

FIG. 4

Growth of the indicated Lactobacillus strains on MRS agar plate supplemented with 10 μg/ml chloramphenicol and in presence or not of 100 ng/ml SpplP.

(a) L. paracasei BL23, (b) L. acidophilus ATCC 4356, (c) L. plantarum NL42.

FIG. 5

Deletion of the upp gene in L. gasseri DSM 14869 by IPSD assisted recombineering.

(a) Single-crossover integration of the upp-Cmr fragment into the chromosome of L. gasseri DSM 14869. Colony PCR using primers pIrec-F and pIrec-R was performed to detect the intact plasmid pINTZrec-upp. Ten colonies without the corresponding band were regarded as candidate single-crossover clones, which were labelled “+”.

(b) The single-crossover event in the ten candidate clones was further confirmed by DNA extraction and PCR using primers uppleft-F and pIrecSC-R (the upper gel), or pIrecSC-F and uppright-R (the lower gel). The correct clones were labelled “+” and the uncertain clones were labelled “?”. (c) Colony PCR using primers uppseq-F and uppseq-R was performed to detect upp double-crossover deletion mutants selected on SDM medium supplemented with 5-FU. M, GeneRuler™ 1 kb DNA ladder; P, DNA of L. gasseri DSM 14869 harboring plasmid pINTZrec-upp; WT, DNA of wild type L. gasseri DSM 14869.

FIG. 6

The inducible promoters used to drive the expression of recombinase are not effective in some strains. Growth of the indicated L. sakei NC03 strain (a) and L. rhamnosus GG strain (b) on MRS agar plate supplemented with 10 μg/ml chloramphenicol and in the presence or absence of 100 ng/ml SppIP.

FIG. 7

Schematic representation of the EPS gene cluster (A) and protein domains of N506_1778 (B) and N506_1709 (C) in L. gasseri DSM 14869. (A) The EPS gene cluster (N506_0396 to N506_0411) which is located nucleotide number 379, 930-394,580 in genome sequence (CP006803) is shown. The enzyme and gene names were annotated by BLASTP analysis using the strains L. gasseri ATCC 33323 and L. gasseri CECT 5714 (Marcotte et al. 2017). (B) The N506_1778 protein includes a YSIRK signal sequence in N-terminus and a Gram-positive LPxTG (LPQTG) motif in C-terminus. The repetitive region consists of two MucBP-like domains. (C) The N506_1709 protein includes a N-terminal YSIRK signal sequence and a C-terminal LPQTG motif. The repeat region harbors three different Rib/alpha-like repeats including one that is only partial. The N-terminus (ca. 1-900 aa) shows no similarities with other proteins in the databank, while the C-terminus (ca. 900-1456 aa) shows low percentage identity (34-48%) with the C-terminus of proteins from L. johnsonii and L. gasseri (accession number WP_095670316, QYS15157, WP_061400034, OYS08635, OYS05727, OUL52955 and AHA97914). Genes and protein structure are not represented at scale.

FIG. 8

(A) Transmission electron microscopy pictures of L. gasseri DSM 14869 wild-type (WT), EPS mutant (ΔEPS) and complemented strains. A dense EPS layer was observed in WT, while the EPS production on the surface was significantly reduced for the EPS mutant strain. The EPS complement strain restored the EPS level to WT. Scale bar=200 nm. (B) Comparison of the thickness of EPS layer of WT, ΔEPS and EPS complement strains. The relative thickness is shown as a percentage relative to WT (set at 100%). Thickness was evaluated in 10 cells for each group and the mean±SD of thickness per cell was determined.

FIG. 9

(A) Phenotypic analysis of L. gasseri DSM 14869 (WT) and its mutant derivatives. The overnight culture in MRS medium was vortexed and put at room temperature for 1 h. The mutant showed a cell sediment and a very clear upper solution. In contrast, a relatively homogenous suspension can be seen for the WT and the complemented strain. (B) Quantitative analysis of auto-aggregation of WT and its mutant derivatives. The overnight cultures were washed and resuspended in PBS to an OD₆₀₀ of 1.5 and were left undisturbed for 5 h. The OD of the upper suspension was measured. The auto-aggregation percentage was calculated by the expression as follows: auto-aggregation (%)=(1−(OD₆₀₀ 5 h/OD₆₀₀ 0 h))×100, where OD₆₀₀ 5 h represents the absorbance at the 5 h time point and OD₆₀₀ 0 h represents the absorbance at 0 h. The auto-aggregation capacities are shown as a percentage relative to wild-type strain (set at 100%). Data represent means±SD of three independent experiments, *P<0.05, ***P<0.001. (C) Quantification of biofilm formation of WT and its mutant derivatives. The biofilms formed on polystyrene plates were assessed after 72 h of incubation in MRS medium using crystal violet staining. The absorbance values at OD₅₇₀ were read and represent the capacity of biofilm formation. Data represent means±SD of three independent experiments, ***P<0.001.

FIG. 10

Comparison of the adhesion ability of L. gasseri DSM 14869 wild-type, EPS mutant and complemented strain. The adhesion rates are shown as a percentage relative to wild-type (set at 100%). Each panel shows the adhesion ability towards (a) Caco-2 cells, (b) HeLa cells, and (c) vaginal epithelial cells. Data represent means±SD of three independent experiments, **P<0.01, ***P<0.001.

FIG. 11

The mRNA expression of genes N506_1778 in WT, N506_1778 mutant (Δ1778) and N506_1778 complement (1778 complement) strains (A) and N506_1709 in WT, N506_1709 mutant (Δ1709) and N506_1709 complement (1709 complement) strains (B). The mRNA expression of the two genes in the WT served as a control. Data represent means±SD of three independent experiments.

FIG. 12

Comparison of the adhesion ability of L. gasseri DSM 14869 wild-type, mutant Δ1778 and Δ1709 strains. The adhesion rates are shown as a percentage relative to wild-type (set at 100%). Each panel shows the adhesion ability towards (a) Caco-2 cells, (b) HeLa cells, (c) vaginal epithelial cells. Data represent means±SD of three independent experiments, *P<0.05, **P<0.01, ***P<0.001.

FIG. 13

(A) The mRNA expression of N506_1709 in overexpressed strain L. lactis NZ9000/pNZ8048-1709. The L. lactis harboring empty pNZ8048 plasmid (NZ9000/pNZ8048) served as a control. (B) Adhesion ability of overexpressed strain NZ9000/pNZ8048-1709 to vaginal epithelial cells. The adhesion rates are shown as a percentage relative to NZ9000/pNZ8048 (set at 100%). Data represent means±SD of three independent experiments, ***P<0.001.

FIG. 14

Growth curve of L. gasseri DSM 14869 wild type, N506_0400 mutant (ΔEPS), N506_1778 mutant (Δ1778) and N506_1709 mutant (Δ1709) strains in MRS medium.

FIG. 15

(A) The plasmid profile of pINTZrec. This plasmid includes a multiple clone site which can be used to integrate homologous fragments; two six sites and a β-recombinase (Rec) that specifically catalyzes the recombination between two six sites that flanked the antibiotic resistance gene and DNA to be integrated. The expression of β-recombinase is controlled by the sakacin-inducible promoter. (B) The plasmid profile of pNZ8048. (C) The plasmid profile of pNZe-Rec.

### DETAILED DESCRIPTION OF THE INVENTION

In a first aspect, the present invention provides a circular DNA vector comprising:
(a) a selectable marker gene sequence, wherein said marker gene sequence is operably linked to a first promoter sequence,
(b) a multiple cloning site, wherein said multiple cloning site optionally comprises a gene targeting sequence,
(c) a sequence encoding a site-specific recombinase, wherein said sequence is operably linked to a second promoter sequence, wherein said second promoter is inducible,
(d) a replicon sequence,
(e) two target sites for said site-specific recombinase, wherein vector comprises a first region flanked on each side by one of said target sites for said site-specific recombinase and wherein said region comprises (a) and (b) with the proviso that (c) and (d) are not within said first region. The circular DNA vector is preferably a plasmid.

The selectable marker may be any marker suitable for identifying and selecting the host cell expressing in the marker. In one embodiment, the selectable marker is an antibiotic resistance gene, such as an antibiotic resistance gene selected from the group consisting of a chloramphenicol resistance gene, spectinomycin resistance gene, tetracycline resistance gene and erythromycin resistance gene.

The site-specific recombinase may be any site-specific recombinase suitable for genetic engineering. In one embodiment, the site-specific recombinase is a site-specific serine recombinases. In another embodiment, the site-specific recombinase is selected from the group consisting of beta-recombinase, Cre-recombinase, FLP-recombinase and PhiC31 integrase.

The target sites for the site-specific recombinase are typically between 30 and 200 nucleotides in length and consist typically of two motifs with a partial inverted-repeat symmetry, to which the recombinase binds, and which flank a central crossover sequence at which the recombination takes place. Examples of target sites includes six (the target site of beta-recombinase), Lox (the target site of the Cre recombinase) and FRT (the target site of the FLP recombinase). The target sites for the site-specific recombinase may be engineered to facilitate the recombination. Sets of single mutant sites may be used, e.g. lox66 and lox71, which produce a double mutant site as a product of the site-specific recombination. The double mutant site is not substrate for the site specific recombinase and thus reversible site-specific recombination events is prevented.

In a preferred embodiment, circular DNA vector comprises two target sites for said site-specific recombinase are orientated such that the product of site-specific recombination between two target sites for said site-specific recombinase is a first circular DNA product comprising (a) and (b) and a second circular DNA product comprising (c) and (d).

The circular DNA vector comprises a replicon, preferably a prokaryotic replicon sequence, which allows the replication of the vector in a cloning host (e.g. E. coli) from which the vector may be harvested. The replicon is useful expansion of the vector and cloning purposes, e.g. cloning a gene targeting sequence. In one embodiment, the replicon sequence is a replicon sequence that allows replication of said vector in E. coli, such as the replication origin of pBR322. The circular DNA vector comprises a replicon that allows replication of the host cell, which is subject to the targeted insertion of the DNA vector (e.g. a Lactobacilli or a Bifidobacteria). In one embodiment, the circular DNA vector comprises a replicon for the replication of the vector in a cloning host and a further replicon for the replication of the vector in the host cell, which is subject to the targeted insertion of the DNA vector in the genome of the host cell. In another embodiment, the replicon sequence is a replicon sequence that allows replication of said vector in E. coli and a further prokaryotic host cell, such as a host cell selected from the group consisting of Lactobacilli and Bifidobacteria. Thus, in this embodiment, the replicon has a dual function, i.e. replication in cloning host cell, for vector expansion and cloning and replication of the vector in the host cell, which is subject to the targeted insertion of the DNA vector in the genome of the host cell.

In one embodiment, the replicon sequence is a replicon sequence that allows replication of said vector in a least one host cell selected from the group consisting of a Lactobacillus gasseri (such as Lactobacillus gasseri DSM 14869), Lactobacillus rhamnosus (such as Lactobacillus rhamnosus DSM 14870), Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

The replicon sequence typically encodes a origin of replication (ori) and replication initiator protein (Rep protein). In one embodiment, the replicon comprises repA encoding Regulatory protein RepA or repB encoding RepFIB replication protein A or RepC encoding Replication initiation protein.

The selectable marker gene sequence is operably linked to a first promoter sequence. The first promoter directing the expression of the selectable marker is preferably a promoter that is constitutively active and thus expressing the marker in the host cell which is subject to the targeted insertion of the DNA vector. Any suitable promoter and marker may be used.

In order to control the timing of the site-specific recombination, the site-specific recombinase is conditionally expressed, e.g. using any suitable inducible promoter. The choice of promoter for expression of the recombinase (the second promoter) depends on the host cell. In one embodiment, the second promoter sequence is an inducible prokaryotic promoter. In another embodiment, the second promoter sequence is selected from the group consisting of a sakacin-inducible promoter, a tetracycline-inducible promoter, D-xylose-inducible promoter, lactose-inducible promoter, IPTG-inducible promoter, nisin inducible promoter, a bile inducible promoter, a bacteriocin-inducible promoter and a synthetic inducible promoter.

The circular DNA vector comprises a cloning site, preferable a multiple cloning site, which allows the cloning of a gene targeting sequence in the vector. In a preferred embodiment, the vector comprises a gene targeting sequence inserted in the multiple cloning site. The gene targeting sequence comprises sequences with sequence identity sufficiently high to allow the targeted integration of the vector at the corresponding target region in the host cell. The circular DNA vector comprising the gene targeting sequence inserted at the cloning site is suitable for use in the methods of the present invention. The host cell is preferably a bacterial cell, more preferably a host cell selected from the group consisting of Lactobacilli and Bifidobacteria. The vector may be introduced in the host cell using any suitable method, such as by transformation, such as electro-transformation, conjugation or transduction.

The circular DNA vector preferably comprises a replicon that allows replication of the vector in the host cell, wherein the vector is to be inserted in the genome by homologous recombination. In the episomal state in the host cell, the vector may be re-arranged by expression of the site-specific recombinase, which (i) eliminates the vectors ability to replicate in the host cell and (ii) facilitates the selection of homologous recombination event between the vector and chromosomal DNA. The product of the site-specific recombination event is a first circular DNA product and a second DNA product, where the first circular DNA product comprises the selectable marker and the multiple cloning site in which the targeting sequence is inserted. The second circular DNA product comprises the replicon and the sequences encoding the site-specific recombinase.

Single cross-over genomic integration of the circular DNA vector (first homologous recombination event) may be selected using the selectable marker. Cells comprising the episomal circular DNA vector is limited due to the expression of the site-specific recombinase that eliminates the vectors ability to replicate in the host cell. Target specific integration of the DNA vector (no longer circular) may be verified e.g. by PCR. The host may be propagated to allow a further (second) homologous recombination event, the product of which is the excision of the selectable marker. The host having undergone the two homologous recombination events may be identified by counterselection for loss of the selectable marker, e.g. sensitivity to the antibiotic where an antibiotic resistance marker is used. The correct double cross-overs should preferably by identified using methods such as PCR and/or sequencing.

A second aspect of the present invention provides a method for introducing recombination between a circular DNA vector and a target region of the genome of a host cell, said method comprising the steps of:
(i) introducing a circular DNA vector comprising a gene targeting sequence according to invention in a host cell, wherein said target sequence comprising flanking sequences comprising at least about 200 consecutive nucleosides having a sequence identity of at least 80% to the corresponding region of the target region of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and
(iv) selecting a host cell, wherein (a) have been excised from the genome of the host cell obtained under (iii) by a second homologous recombination event between the flanking sequences of the targeting sequence and the target region of the genome.

The circular DNA vector of the present invention may be used to introduce a specific mutation in a target region of a host cell. The target region may be a coding region, such as a gene encoding a protein. The mutation may be a point mutation that change or disrupt the encoded sequence. The encoded sequence may also be disrupted by mutations that introduces a deletion or insertion in the sequence. Alternatively, the mutation may change (decrease or increase) or restore the activity of the encoded protein.

Accordingly, a third aspect of the present invention provides a method for generating a host cell having a mutation in a target gene of the genome of a host cell, said method comprising the steps of:
(i) introducing a circular DNA vector comprising a gene targeting sequence according to invention in a host cell, wherein said target sequence comprising flanking sequences comprising at least about 200 consecutive nucleosides having a sequence identity of at least 80% to the corresponding region of the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and (iv) selecting a host cell, wherein (a) have been excised from the genome of the host cell obtained under (iii) by a second homologous recombination event between the flanking sequences of the targeting sequence and the target region of the genome and, wherein said host cell comprises a mutation in said target region.

The method is useful for e.g. evaluation of the function of specific genes. For example, to evaluate if a specific gene has a function e.g. in bacterial adherence, auto-aggregation and/or biofilm formation.

In a related aspect, the invention provides a method for generating a host cell having loss of function in a target gene of the genome of a host cell, said method comprising the steps of:
(i) introducing a circular DNA vector comprising a gene targeting sequence according to invention in a host cell, wherein said target sequence comprising flanking sequences comprising at least about 200 consecutive nucleosides having a sequence identity of at least 80% to the corresponding region of the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and (iv) selecting a host cell, wherein (a) have been excised from the genome of the host cell obtained under (iii) by a second homologous recombination event between the flanking sequences of the targeting sequence and the target region of the genome and, wherein said host cell comprises a loss of function of said target gene.

This method is useful for e.g. evaluation of the function of specific genes as described above. Functional assay may be used to address the potential impact of the targeted engineering at the targeted region resulting in the loss of gene function on phenotypical characteristics of the host cells (compared to the wild-type host cell). Phenotypical characteristics include, but are not limited to bacterial adherence (e.g. to mammalian tissue), auto-aggregation and/or biofilm formation.

Another aspect of the present invention provides a method for generating a host cell having gain of function in a target gene of the genome of a host cell, said method comprising the steps of:
(i) introducing a circular DNA vector comprising a gene targeting sequence according to invention in a host cell, wherein said target sequence comprising flanking sequences comprising at least about 200 consecutive nucleosides having a sequence identity of at least 80% to the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and (iv) selecting a host cell, wherein (a) have been excised from the genome of the host cell obtained under (iii) by a second homologous recombination event between the flanking sequences of the targeting sequence and the target region of the genome and, wherein said host cell comprises a gain of function of said target gene.

This method is useful for e.g. evaluation of the reversion of a previous loss of gene function in a host cell restore lost phenotypical characteristics such as bacterial adherence (e.g. to mammalian tissue), auto-aggregation and/or biofilm formation.

The target gene may encode any gene product. The target gene typically encodes a protein. In one embodiment, the target gene encodes a cell surface protein. In one embodiment, the target gene encodes a protein involved in bacterial adherence, auto-aggregation and/or biofilm formation. In another embodiment, the cell surface protein is a sortase dependent protein (SDP) or a S-layer protein or encodes a protein involved in the biosynthesis of a cell surface molecule. In one embodiment, the cell surface molecule is an exopolysaccharide (EPS). In a particular embodiment, the target gene is a gene selected from the group consisting of N506_1709, N506_1778, N506_0396, N506_0397, N506_0398, N506_0399, N506_0400, N506_0401, N506_0402, N506_0403, N506_0404, N506_0405, N506_0406, N506_0407, N506_0408, N506_0409, N506_0410 and N506_0411.

In one embodiment, the host cell is a Lactobacillus gasseri (such as Lactobacillus gasseri DSM 14869) and the target gene is selected from the group consisting of N506_1709, N506_1778, N506_0396, N506_0397, N506_0398, N506_0399, N506_0400, N506_0401, N506_0402, N506_0403, N506_0404, N506_0405, N506_0406, N506_0407, N506_0408, N506_0409, N506_0410 and N506_0411.

The circular DNA vector of the present invention may be used to prepare host cell recombinant expression of recombinant gene products, such as recombinant proteins. In this aspect, the gene targeting sequence will typically comprises sequences having sufficient sequence identity to the targeting region in order to allow target region specific integration; and a sequence encoding the recombinant gene product, e.g. a polypeptide.

Accordingly, in a further aspect, the present invention provides a method for preparing a host cell expressing a recombinant polypeptide, said method comprising the steps of:
(i) introducing a circular DNA vector comprising a gene targeting sequence according to invention in a host cell, wherein said target sequence comprising flanking sequences comprising at least about 200 consecutive nucleosides having a sequence identity of at least 80% to the corresponding region of the target region of the host cell genome, and wherein said gene targeting sequence encodes a recombinant polypeptide,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the target sequence and the target region of the genome of said host cell, and
(iv) selecting a host cell, wherein (a) have been excised from the genome of the host cell obtained under (iii) by a second homologous recombination event between the flanking sequences of the targeting sequence and the target region of the genome.

In one embodiment, the recombinant polypeptide is selected from the group consisting of antibodies (such as monoclonal antibodies, humanized monoclonal antibodies, chimeric antibodies, single-domain antibodies, camelid antibodies), enzymes, cytokines, hormones and blood-clotting proteins. The host cell obtained by the method may be used as a producer cell for production of the recombinant product.

The gene targeting sequence comprises sequences in the flanking region that has sufficiently high identity to the sequence of the target region in order to allow/facilitate homologous recombination. In one embodiment, the sequences in the flanking region (the flanking sequences) comprises consecutive nucleosides in the range of 200 to 1500, for example at least about 300 consecutive nucleosides, such as at least about 400 consecutive nucleosides, for example at least about 500 consecutive nucleosides, such as at least about 600 consecutive nucleosides, for example at least about 700 consecutive nucleosides, such as at least about 800 consecutive nucleosides, for example at least about 900 consecutive nucleosides, such as at least about 1000 consecutive nucleosides, for example at least about 1100 consecutive nucleosides, such as at least about 1200 consecutive nucleosides, for example at least about 1300 consecutive nucleosides, such as at least about 1400 consecutive nucleosides, for example at least about 1500 consecutive nucleosides.

In another embodiment, the flanking sequences have a sequence identity of at least 85% to the corresponding region of the target gene of the host cell genome, for example at least 95% to the corresponding region of the target gene of the host cell genome, such as at least 97% to the corresponding region of the target gene of the host cell genome, for example at least 98% to the corresponding region of the target gene of the host cell genome, such as at least 99% to the corresponding region of the target gene of the host cell genome, for example 100% to the corresponding region of the target gene of the host cell genome.

In one embodiment, the selection under (iii) uses the selectable marker gene sequence (a) of the circular DNA vector (which selects for host cells having a single cross-over integration of the DNA vector). Host cells comprising a single cross-over integration of the DNA vector may be identified/verified using PCR and/or DNA sequencing. Thus in one embodiment, the selection under (iii) uses PCR and/or DNA sequencing, such as sequencing the sequence junction between the targeting sequence and the target region.

The host may be propagated to allow a further (second) homologous recombination event, the product of which is the excision of the selectable marker. The host having undergone the two homologous recombination events may be identified by counterselection for loss of the selectable marker, e.g. resulting in sensitivity to the antibiotic where an antibiotic resistance marker is used. The correct double cross-overs events should preferably by identified using methods such as PCR and/or DNA sequencing. In a preferred embodiment, the host cells having undergone a first and a second homologous recombination event are selected and/or verified using PCR or DNA sequencing.

The product of the method of the invention (the product of (iv)) is typically a host cell comprising a deletion of the target region, a partial deletion of the target region, a sequence insertion at the target region, a point mutation of the target region, or a sequence replacement of the target region.

The host cell is preferably a prokaryote, preferably a bacteria. In a preferred embodiment, the host cell is a Lactobacilli or Bifidobacteria. In another embodiment, the host cell is selected from the group consisting of a Lactobacillus gasseri (such as Lactobacillus gasseri DSM 14869), Lactobacillus rhamnosus (such as Lactobacillus rhamnosus DSM 14870), Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii. In a preferred embodiment, the host cell is a Lactobacillus gasseri (such as Lactobacillus gasseri DSM 14869).

In a further aspect, the present provides a recombinant host cell obtainable by the method of the present invention. In one embodiment, the host cell is a Lactobacillus or Bifidobacteria. In another embodiment, the host cell is a Lactobacillus gasseri (such as Lactobacillus gasseri DSM 14869). In another embodiment, the host cell is a Lactobacillus gasseri (such as Lactobacillus gasseri DSM 14869) engineered to express a gene selected from the group consisting of N506_1709, N506_1778, N506_0396, N506_0397, N506_0398, N506_0399, N506_0400, N506_0401, N506_0402, N506_0403, N506_0404, N506_0405, N506_0406, N506_0407, N506_0408, N506_0409, N506_0410 and N506_0411. In a preferred embodiment, the Lactobacillus gasseri (such as Lactobacillus gasseri DSM 14869) is engineered to express N506_1709 and/or N506_1778,

In one embodiment, the coding region of the gene is operably linked to a recombinant promoter, preferably a recombinant constitutive promoter.

One aspect of the present provides the use of the circular DNA vector of the present invention for introducing a gene sequence in the genome of a host cell. In one embodiment, the use of the circular DNA vector is for increasing tissue adhesion of a host cell, such as for increasing tissue adhesion of a bacterial host cell to vaginal tissue, preferably human vaginal tissue. The host cell is preferably a Lactobacilli or Bifidobacteria. In one embodiment, the host cell selected from the group consisting of a Lactobacillus gasseri (such as Lactobacillus gasseri DSM 14869), Lactobacillus rhamnosus (such as Lactobacillus rhamnosus DSM 14870), Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

In one embodiment, the use of the circular DNA vector is for introducing a deletion of a target region, partial deletion of a target region, a sequence insertion at a target region, a point mutation of a target region, or a sequence replacement of a target region.

In one embodiment, the use of the circular DNA vector is for targeting a target gene selected from the group consisting of N506_0396, N506_0397, N506_0398, N506_0399, N506_0400, N506_0401, N506_0402, N506_0403, N506_0404, N506_0405, N506_0406, N506_0407, N506_0408, N506_0409, N506_0410 and N506_0411 in a Lactobacillus gasseri (such as Lactobacillus gasseri DSM 14869) host cell.

In another embodiment, the use of the circular DNA vector is for introducing and expression of said gene sequence in said host cell. In yet another embodiment, said gene sequence blocks expression of an endogenous host gene. In a further embodiment, the gene sequence replaces a corresponding endogenous host gene.

In one embodiment, the use of the circular DNA vector is for increasing tissue adhesion of a bacterial host cell, such as increasing tissue adhesion of a bacterial host cell (such as a Lactobacilli or Bifidobacteria) to vaginal tissue, preferably human vaginal tissue. In one embodiment, the host cell is selected from the group consisting of a Lactobacillus gasseri (such as Lactobacillus gasseri DSM 14869), Lactobacillus rhamnosus (such as Lactobacillus rhamnosus DSM 14870), Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

### EXAMPLES

### Example 1

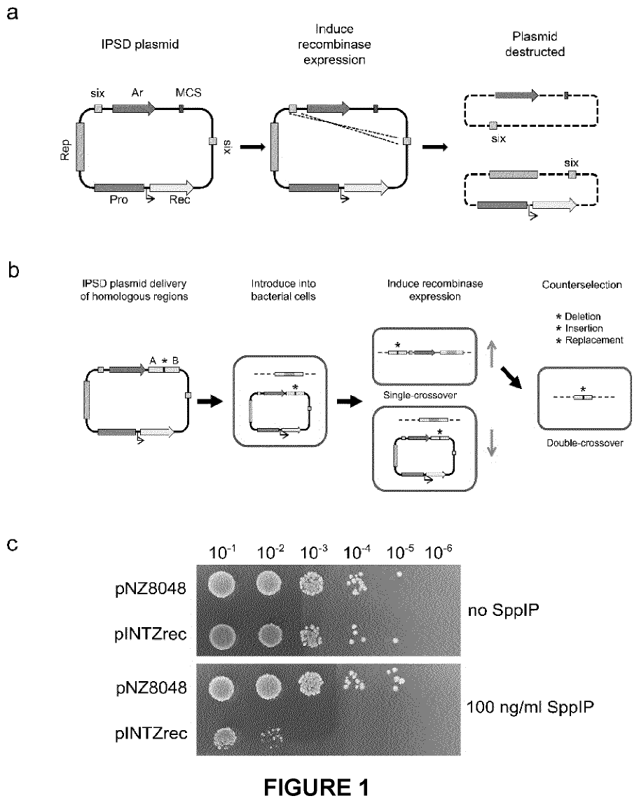

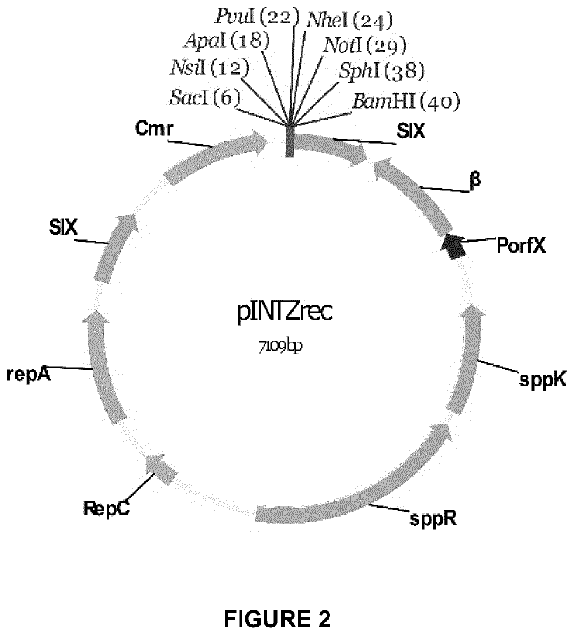

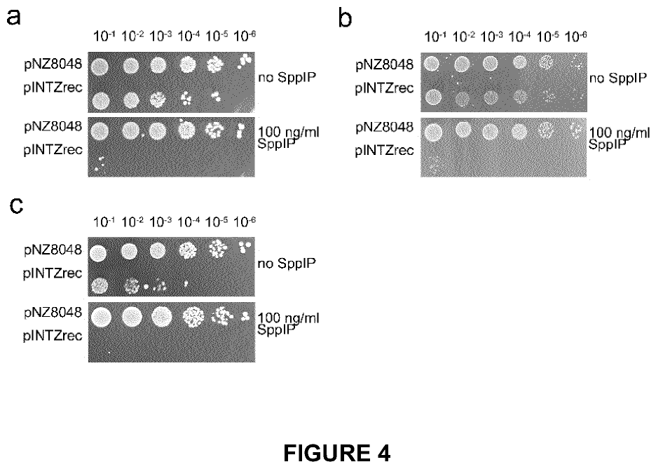

Provided is a novel vector, which may be conditionally destructed facilitating the selection of homologous recombination event between the vector and chromosomal DNA. Essentially, the replicon and the antibiotic resistance gene were separated by two oriented six fragments. Upon addition of the inducer and expression of the site-specific recombinase (β), the vector can recombine and lose function due to the excision of the replicon (FIG. 1a). This vector termed Inducible Plasmid Self-Destruction (IPSD) could be used to assist bacterial recombineering, including gene deletion, insertion, and replacement (FIG. 1b). As a proof-of-concept, an IPSD plasmid pINTZrec was constructed based on the β-six recombination system (FIG. 2) in which the β-recombinase gene was under the control of the sakacin-inducible promoter P_{orfX}. When the plasmid pINTZrec carrying a chloramphenicol resistance gene was introduced into the vaginal probiotic strain L. gasseri DSM 14869, the pINTZrec transformant showed a dramatic sensitivity to Sakacin P (SpplP) induction. The viability of SpplP-induced pINTZrec transformant on MRS agar plates supplemented with chloramphenicol was decreased by three to four orders of magnitude compared to the non-induced control (FIG. 1c), suggesting that the plasmid pINTZrec was destructed following expression of β-recombinase. Such observation was also found in other Lactobacillus species (L. paracasei, L. acidophilus, L. plantarum) (FIG. 4).

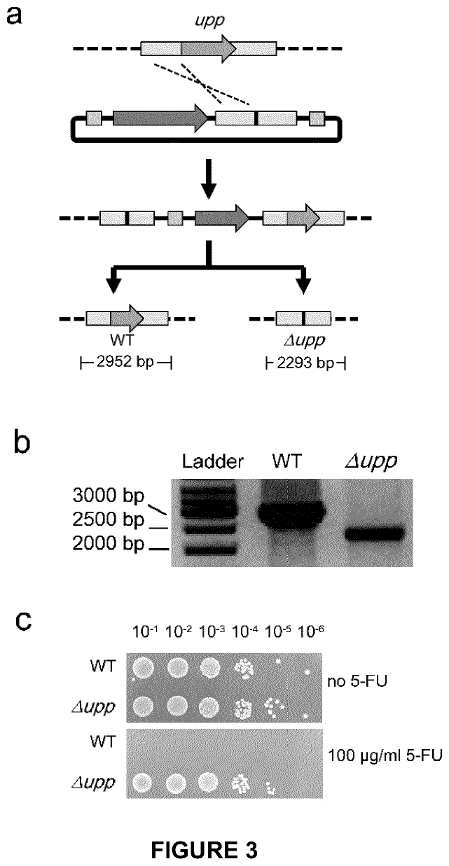

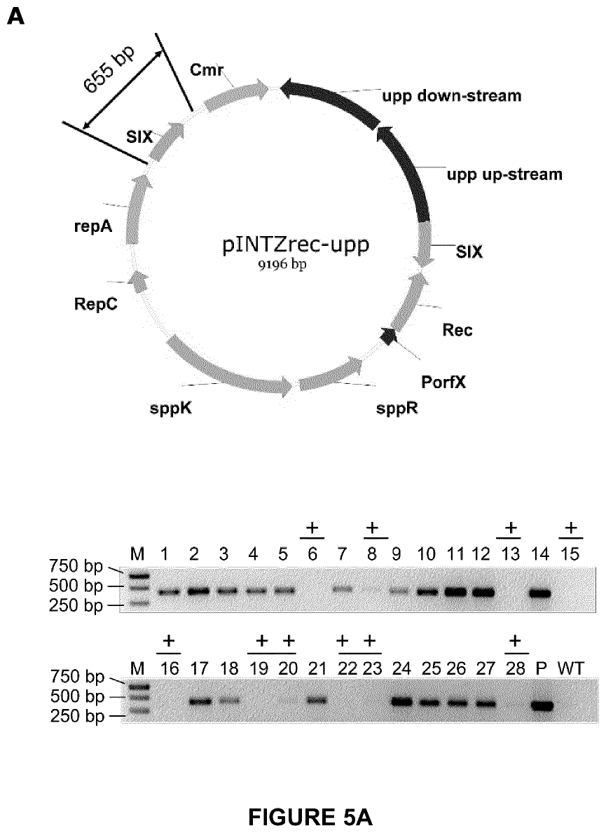

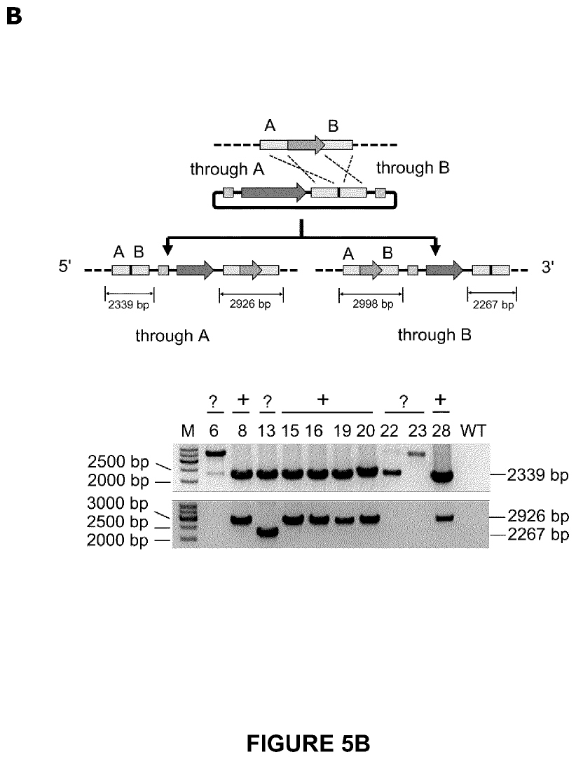

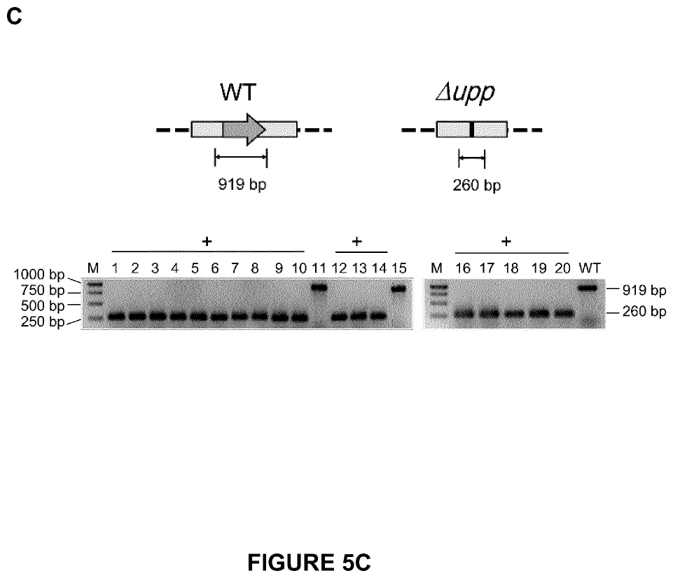

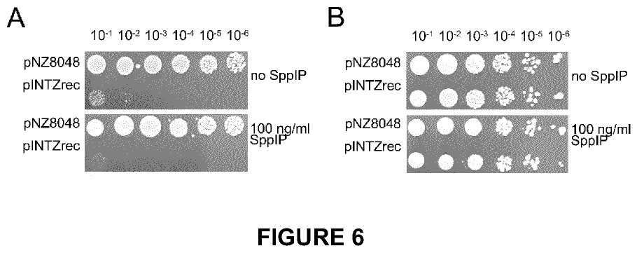

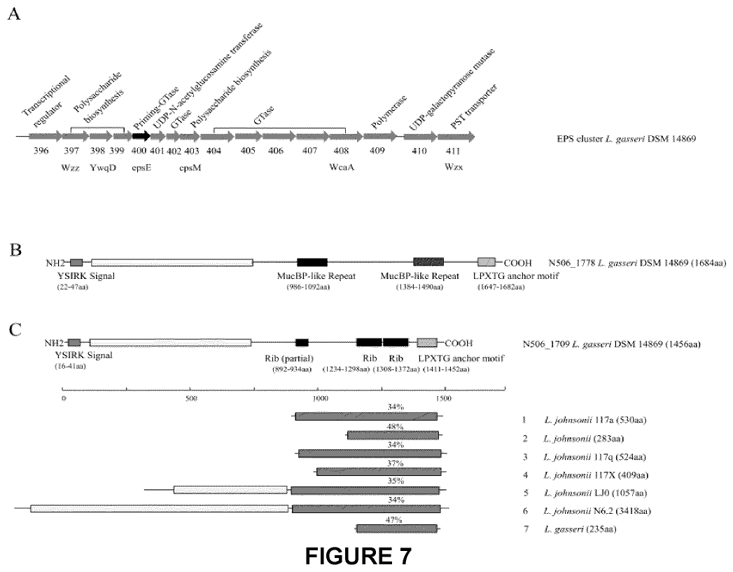

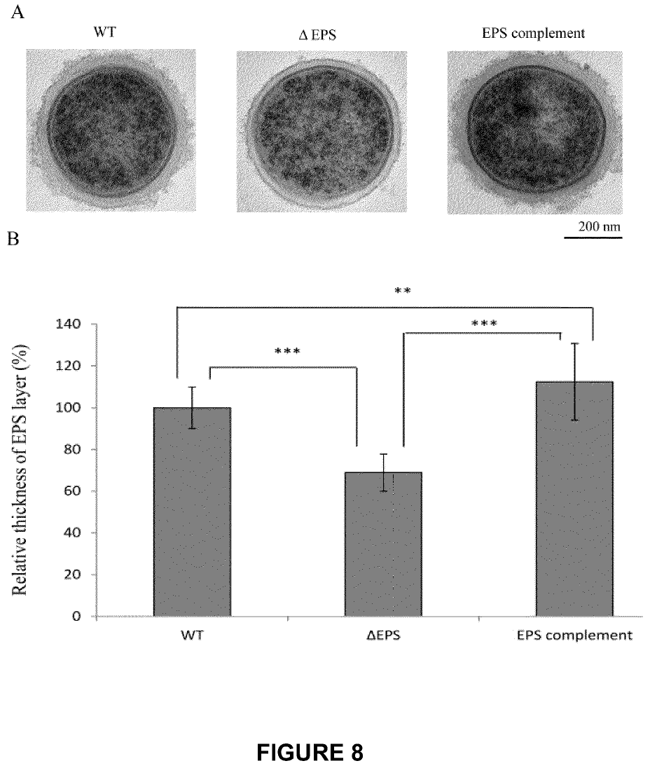

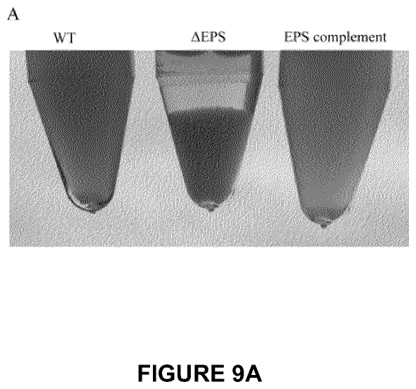

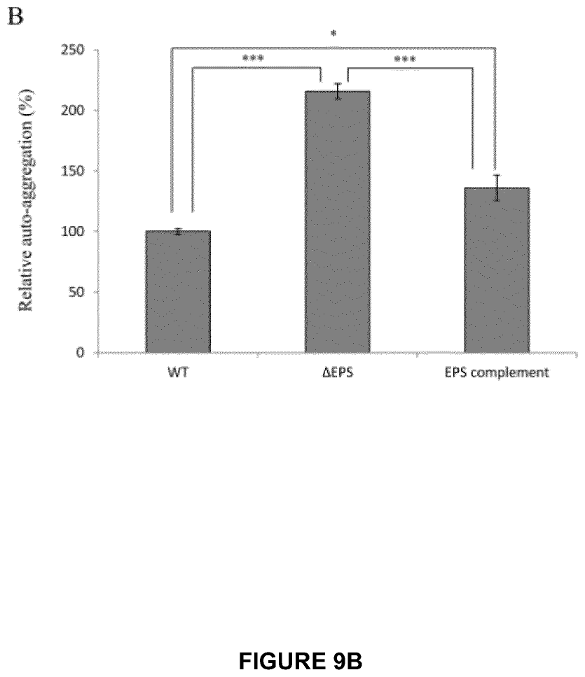

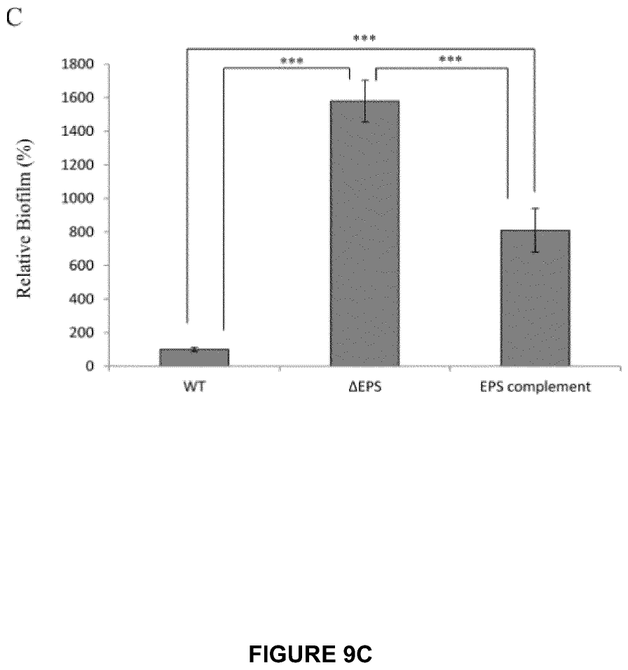

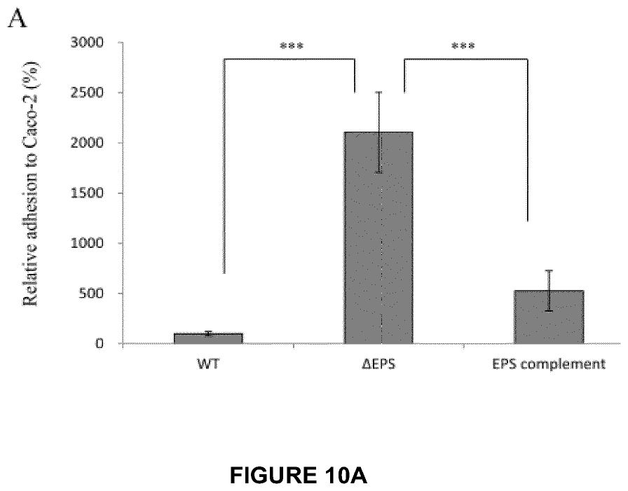

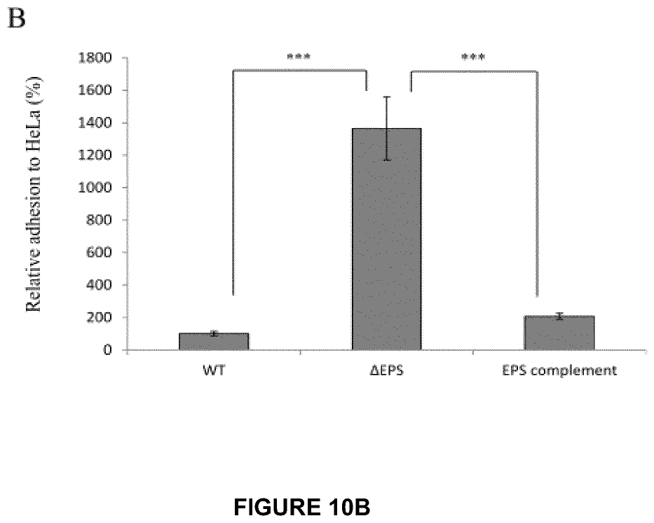

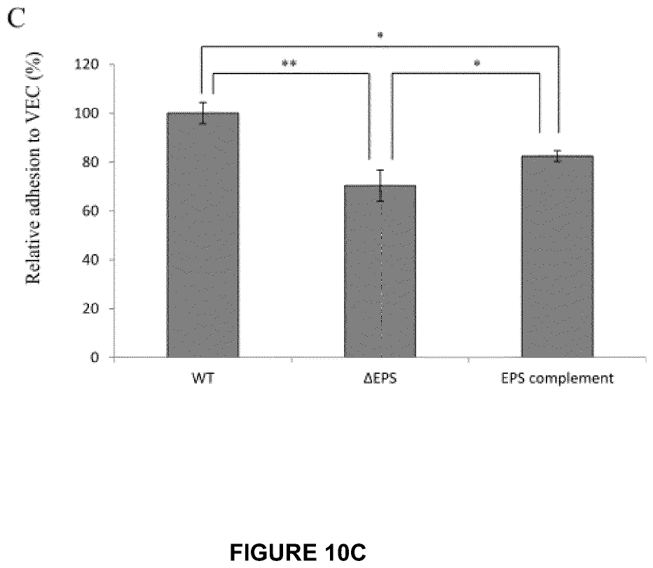

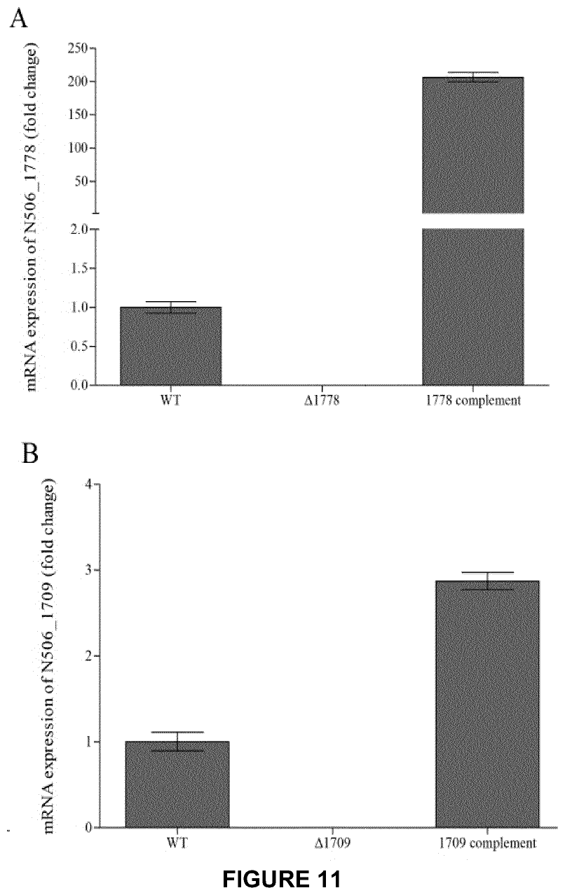

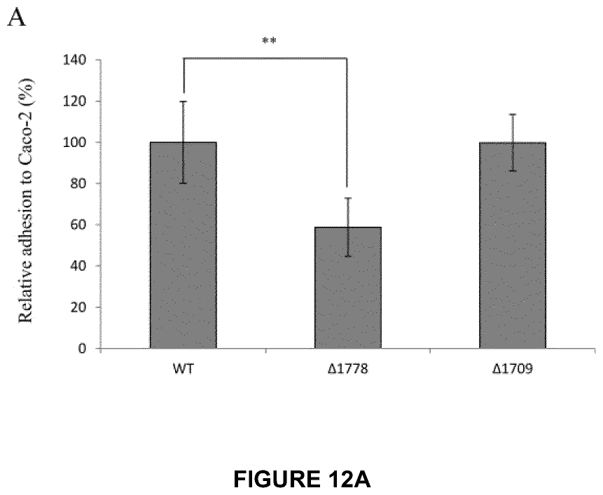

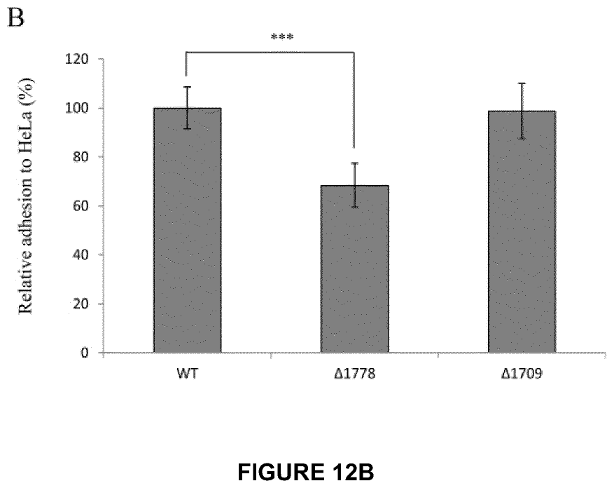

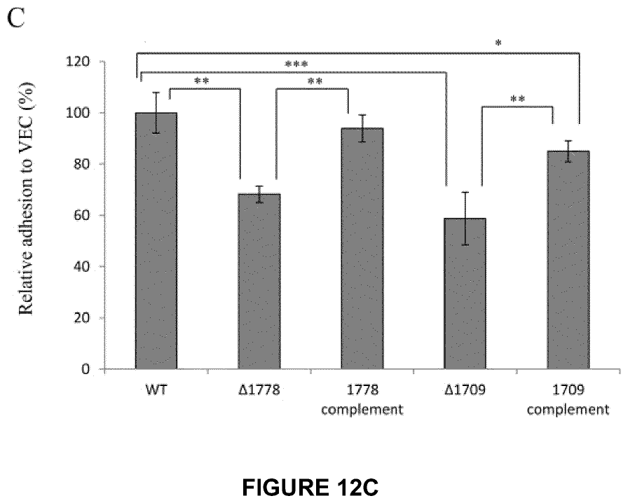

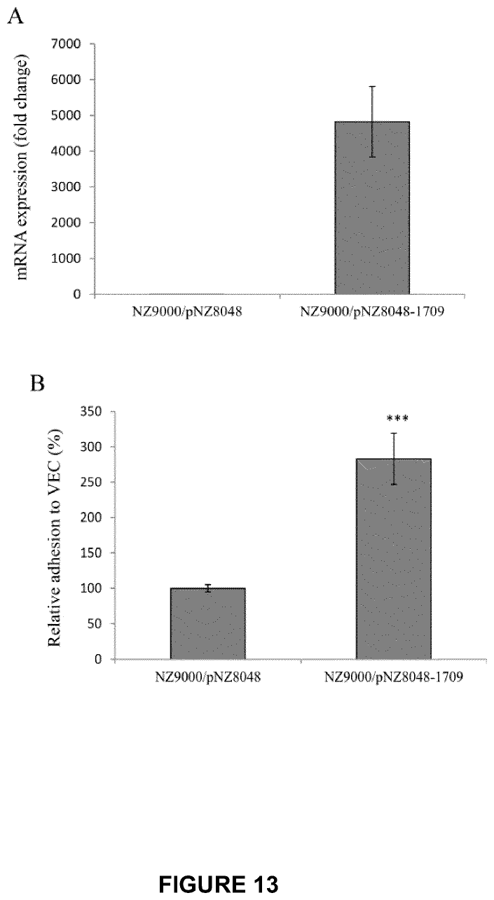

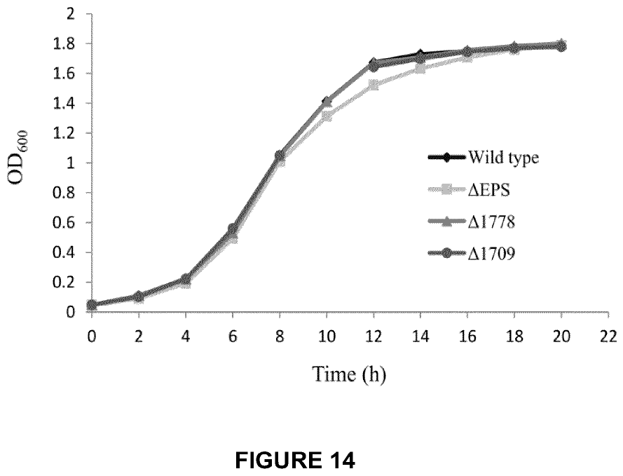

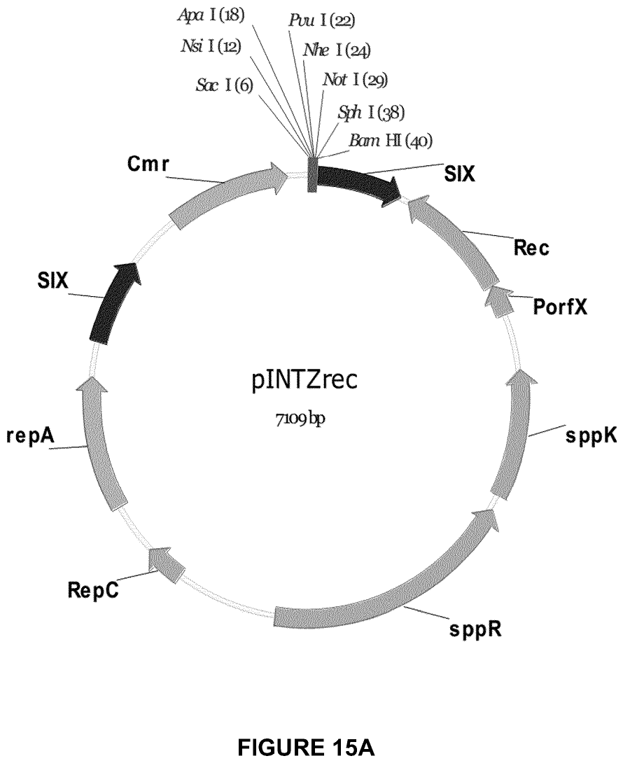

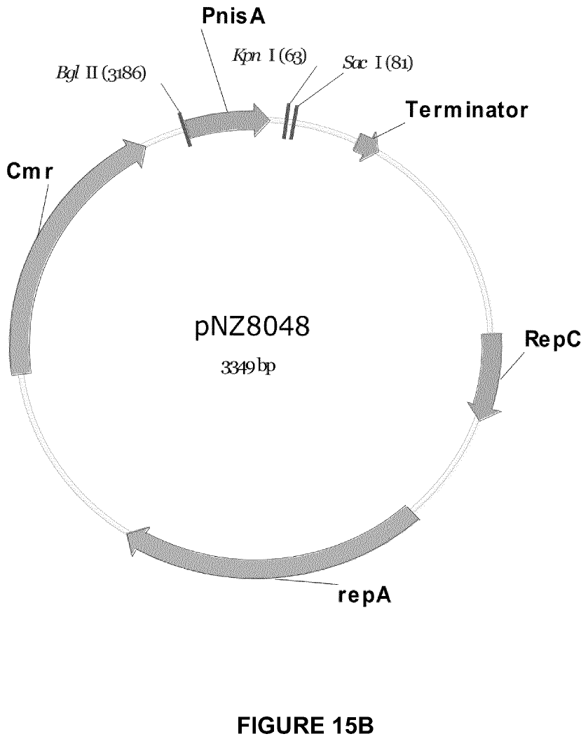

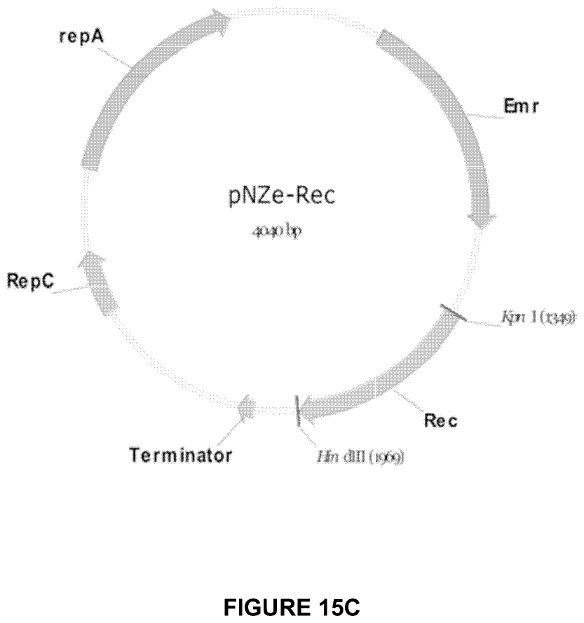

Previous work by the inventors showed that the transformation efficiency of L. gasseri DSM 14869 was very low possibly due to the thickness of EPS covering the cell surface or the presence of two resident plasmids, and therefore, generating mutation in this strain using existing methods was not possible (Marcotte, H. et al. (2017) and unpublished data). In order to demonstrate IPSD assisted recombineering in L. gasseri DSM 14869, we targeted upp, a non-essential gene encoding uracil phosphoribosyltransferase (UPRTases) that is commonly used as a counterselection marker. A recombinant plasmid pINTZrec-Rupp containing homologous regions up- and downstream of upp gene was constructed and introduced into L. gasseri DSM 14869. The transformant was induced by SpplP and the single-crossover integration event was selected using colony PCR (FIG. 3a, FIG. 5a). Ten out of randomly picked 28 (36%) colonies showed absence of plasmid pINTZrec-Rupp, suggesting that the Cm′ expression cassette was integrated into the chromosome of L. gasseri DSM 14869 by a single-crossover event. These results were further confirmed by PCR on DNA extracted from isolated clones, six clones showed correct integration (FIG. 5b). Following growth of the single crossover clones in the absence of antibiotics, the double-crossover upp mutant could easily be selected by counterselection of Cm^{s} colonies or by selection of colonies resistant to 5-Fluorouracil (5-FU) (FIG. 3b, FIG. 5c). The L. gasseri DSM 14869 upp mutant showed resistance to 5-FU (100 μg/ml) in contrast to the parent strain due to the abolished conversion of 5-FU into cell toxic 5-fluorodeoxyuridine monophosphate (5-FdUMP) (FIG. 3c). We have also generated several mutants of cell surface property related genes and integrative expression of broad and potent HIV-1-neutralizing antibodies in L. gasseri DSM 14869 based on this method (unpublished data). These results suggest that the IPSD plasmid could efficiently be used for genome engineering in lactobacilli.

The major advantage of IPSD assisted bacterial recombineering is that it does not depend on transformation (or conjugation) efficiency. However, it needs two pre-requirements: 1) a functional replicon allowing the plasmid to replicate in the host bacteria; 2) a tightly controlled expression element used to drive recombinase gene expression. The inducible promoters used in this study are not effective in some strains, either due to strong background expression (L. sakei NC03) (FIG. 6a) or low induced expression (L. rhamnosus GG) (FIG. 6b). Therefore, the development of a universal tightly controlled expression element for lactobacilli, such as tetracycline-regulated systems, is a desire.

In conclusion, the inventors have shown that the IPSD plasmid can be used for genome engineering in lactobacilli, with the potential to be extend to other bacterial species. The IPSD strategy could be used in a range of applications in both the food and pharmaceutical industry such as identification of probiotic genes, metabolic engineering and delivery of therapeutics, thus opening new avenues for the engineering of biotherapeutic agents with enhanced health-promoting functional features.

Methods Bacterial strains, plasmids and growth conditions Bacterial strains and plasmids used in this study are listed in Table1. Lactobacillus strains were generally cultured at 37° C. in deMan Rogosa Sharpe (MRS) medium (Difco, BD BioSciences). For upp-based double-crossover event selection, the Lactobacillus strain was grown on semi-defined medium (SDM) agar plates (Kimmel et al.). Escherichia coli strains were cultured in Luria-Bertani broth at 37° C. with rotary shaking at 200 rpm, or on LB agar plates. When needed, antibiotics were supplemented at the following concentrations: 10 μg/ml chloramphenicol for Lactobacillus strains and E. coli VE7108 strain.

Plasmid Construction

The primers used in this study are listed in Table 2. For construction of IPSD vector pINTZrec used for lactobacilli recombineering, the two six DNA fragments were amplified from site-specific integration vector pEM76 by using the primers pairs SIX-F1&R1 and SIX-F2&R2, respectively, and inserted into plasmid pNZ8048 on each flank of the Cm′ expression cassette generating pNZ8048-SIX. The orientation of the insertion was confirmed by sequencing. A multiple cloning sites was introduced into pNZ8048-SIX by inserting a linker between PstI and BgIII, resulting in the plasmid pNZmcs-SIX. The β-recombinase gene was amplified from plasmid pEM94 and inserted into plasmid pVPL3017 downstream of the sakacin-inducible promoter P_{orfX} generating pVPL3017-rec. Subsequently, the P_{orfX}-rec expression cassette was digested using SalI and HindIII, and inserted into similarly digested pNZmcs-SIX plasmid to obtain the final plasmid pINTZrec.

For construction of the plasmid used for L. gasseri DSM 14869 upp gene deletion, two 1090 bp DNA fragments, upstream and downstream of upp gene, were amplified from genomic DNA of L. gasseri DSM 14869 using the primer pairs upp-up-F/upp-up-R and upp-down-F/upp-down-R, respectively. The upstream DNA fragment was inserted into pMD19-T Simple vector by TA cloning generating pMD19-upp-up, then the downstream DNA fragment was inserted into pMD19-upp-up between SacI and SphI, generating pMD19-4upp. Δupp was digested with Δpal and SphI and inserted into similarly digested pINTZrec generating pINTZrec-Δupp.

Transformation

The plasmids pINTZrec and pINTZrec-Δupp were electrotransformed into L. gasseri DSM 14869 and other Lactobacillus strains according to De Keersmaecker et al. The plasmid pNZ8048 was used as controls. The transformants were confirmed by colony PCR followed by PCR on DNA extracted from pure cultures.

Recombineering

The single colonies of L. gasseri DSM 14869 harboring the plasmid pINTZrec or its derivatives were grown overnight in MRS broth containing 10 μg/ml chloramphenicol. The cultures were inoculated (1%, v/v) into MRS broth without antibiotics and grown at 37° C. until OD₆₀₀ₙₘ reached ˜0.30, then supplemented with 100 ng/ml sakacin P (SpplP) (Genscript). The cultures were allowed to grow overnight, and serial dilutions were plated on MRS agar supplemented with 10 μg/ml chloramphenicol and 100 ng/ml SpplP. The single-crossover events were detected by colony PCR followed by PCR on DNA extracted from pure cultures. For double-crossover selection, the single-crossover clones were grown overnight in MRS broth in absence of antibiotics, followed by spreading serial dilutions on MRS agar or SDM agar supplement of 100 μg/ml 5-Fluorouracil (5-FU). The colonies from MRS agar were replicated to MRS agar containing 10 μg/ml chloramphenicol, the Cm^{s} colonies were selected and detected by PCR on extracted DNA.

All the mutants generated in this study were confirmed by PCR and sequencing.

TABLE 1
Strains and plasmids.

| Strain or plasmid | Characteristics^{a} | Source |
|---|---|---|
| Strains | | |
| E. coli DH5a | fhuA2 Δ(argF-lacZ)U169 phoA | Invitrogen |
| | glnV44 Φ80 Δ(lacZ)M15 gyrA96 | |
| | recA1 relA1 | |
| | endA1 thi-1 hsdR17 | |
| E. coli VE7108 | Km^{r}, host of pNZ8048 | Turroni, F. et al. |
| L. gasseri DSM 14869 | Human vaginal isolate | Cano-Garrido et |
| | | al. |
| L. paracasei BL23 | Origin unclear | Fukiya et al. |
| L. plantarum NL42 | Cheese isolate | Biswas et al. |
| L. acidophilus ATCC | Human pharynx isolate | Canosa et al. |
| 4356 | | |
| L. sakei NC03 | Sausage isolate | This study |
| L. gasseri DSM 14869 | L. gasseri DSM 14869 upp | This study |
| Δupp | deletion mutant | |
| Plasmids | | |
| pNZ8048 | Cm^{r}, SH71 replicon plasmid | Marcotte, H. et |
| | | al. (2017) |
| pEM76 | Source of six sequence | Goh et al. |
| pEM94 | Source of β-recombinase gene | Lim et al. |
| pVPL3017 | Cm^{r}, pSIP411 derivative, | Krüger, C. et al. |
| | containing sakacin-inducible | |
| | expression element | |
| pVPL3017-rec | pVPL3017 derivative, with | This study |
| | recT1 replaced with β- | |
| | recombinase gene | |
| pNZ8048-SIX | pNZ8048 derivative containing | This study |
| | two six DNA fragments flanking | |
| | Cm^{r} gene | |
| pNZmcs-SIX | pNZ8048-SIX derivative | This study |
| | containing a multiple-cloning | |
| | site | |
| pINTZrec | pNZmcs-SIX derivative | This study |
| | containing β recombinase gene | |
| | expression cassette | |
| pINTZrec-Δupp | pITZrec derivative containing | This study |
| | upstream and downstream DNA | |
| | of L. gasseri DSM 14869 upp | |
| | gene | |

^{a}Km^{r}, kanamycin resistance; Cm^{r}, chloramphenicol resistance; Sm^{r}, spectinomycin resistance.

TABLE 2
Oligonucleotides used in this study.

| Oligo- | | Restriction |
|---|---|---|
| nucleotides | Sequence, 5′-3′^{a} | sites |
| SIX-F1 | GACCGGTCGACAATTATTAGGGGGAGAAG (SEQ | SalI |
| | ID NO: 1) | |
| SIX-R1 | CGCCAGTCGACGAGTCGTGCATAACCAAT (SEQ | SalI |
| | ID NO: 2) | |
| SIX-F2 | GACCGCTGCAGAATTATTAGGGGGAGAAG (SEQ | PstI |
| | ID NO: 3) | |
| SIX-R2 | CGCCAAAGCTTGAGTCGTGCATAACCAAT (SEQ | HindIII |
| | ID NO: 4) | |
| linker-F | GATCTGAGCTCATGCATGGGCCCGATCGCTAGCG | |
| | GCCGCATGCGGATCCTGCA (SEQ ID NO: 5) | |
| linker-R | GGATCCGCATGCGGCCGCTAGCGATCGGGCCCA | |
| | TGCATGAGCTCA (SEQ ID NO: 6) | |
| upp-up-F | TAATTGGGCCCAAATAATGGAAACTAAGATT (SEQ | ApaI |
| | ID NO: 7) | |
| upp-up-R | GTTCAGCATGCTAACAAGAGCTCAGATAAATGTTT | SphI, SacI |
| | CTTAAATCGT (SEQ ID NO: 8) | |
| upp-down-F | CAGGAGAGCTCTTGTTCGGATCCAAGTAATTTTAC | SacI, |
| | TCAAAAATCT (SEQ ID NO: 9) | BamHI |
| upp-down-R | TTACAGCATGCAAAACGCAAATTACAGGAAGAG | SphI |
| | (SEQ ID NO: 10) | |
| uppleft-F | TTACCAGATTTTGAAATTGAGTT (SEQ ID NO: 11) | |
| uppright-R | AGTAAAGCGTATCTCCTAACTCT (SEQ ID NO: 12) | |
| plrec-F | AGATTTATTGAGAGGAGGGATTATT (SEQ ID NO: | |
| | 13) | |
| plrec-R | CGTTTGTTGAACTAATGGGTGCT (SEQ ID NO: 14) | |
| plrecSC-F | AAAGTTTTCGGGCTACTCTCTCCT (SEQ ID NO: | |
| | 15) | |
| plrecSC-R | GGAATTGTCAGATAGGCCTAATGACT (SEQ ID NO: | |
| | 16) | |
| uppseq-F | GAACAATTAGTCCTGCTTATATG (SEQ ID NO: 17) | |
| uppseq-R | CGACTACAGATTTCTCATTCACT (SEQ ID NO: 18) | |

^{a}The restriction sites are underlined.

### Example 2

Lactobacilli play an important role for the maintenance of a healthy vaginal microbiota, and some select species are widely used as probiotics. Vaginal isolates of Lactobacillus gasseri DSM 14869 and L. rhamnosus DSM 14870 were previously selected to develop the probiotic EcoVag® capsules and were shown to have therapeutic effects in women with bacterial vaginosis (BV). However, the molecular basis and mechanisms involved in their probiotic activity are largely unknown. In this study, we identified three cell surface molecules in L. gasseri DSM 14869 that promote adhesion to vaginal epithelial cells by constructing dedicated knock-out mutants, including exopolysaccharides (EPS), a protein containing MucBP-like domains (N506_1778) and a putative novel adhesin (N506_1709) with Rib/alpha-like domain repeats. EPS knock-out mutants revealed a 20-fold and 14-fold increase in adhesion to Caco-2 and HeLa cells, respectively, compared to wild-type, while the adhesion to vaginal cells was reduced 30% by the mutation, suggesting that EPS might mediate tissue tropism for vaginal cells. A significant decrease in adhesion to Caco-2, HeLa and vaginal cells was observed in the N506_1778 knock-out mutant. The N506_1709 mutant showed no significant difference for the adhesion to Caco-2 and HeLa cells compared to WT; in contrast, the adhesion to vaginal cells revealed a significant decrease (42%), suggesting that N506_1709 might mediate specific binding to stratified squamous epithelial cells and this putative novel adhesin was annotated as Lactobacillus vaginal epithelium adhesin (LVEA). Thus, for the first time, the inventors have discovered an important role for EPS and a novel adhesin LVEA in the adhesive capacity of a vaginal probiotic Lactobacillus strain.

Lactobacilli are known to contribute to the maintenance of a healthy vaginal microbiota and some are selected as probiotics for prevention or treatment of urogenital diseases such as bacterial vaginosis. However, the molecular mechanisms for these health-promoting effects are poorly understood. Here, we functionally identified three cell surface factors of a Lactobacillus gasseri strain potentially involved in its adhesion to vaginal epithelial cells, including exopolysaccharides (EPS) and two sortase-dependent proteins (N506_1778 and N506_1709). The inventors have demonstrated for the first time the tissue specific adhesion of EPS to vaginal cells and that N506_1709 might be a novel adhesin specifically mediating bacterial binding to stratified squamous epithelial cells. The results provide important new information on the molecular mechanisms of vaginal Lactobacillus adhesion.

### Introduction

The vaginal microbiota of a healthy woman is usually dominated by lactobacilli and the most frequently occurring species are Lactobacillus crispatus, L. gasseri, L. jensenii, L. vaginalis, and L. iners (Pendharkar et al. 2013; Ravel et al. 2011; Vasquez et al. 2002). These bacteria maintain the normal vaginal microbiota by adhering to vaginal epithelial cells (VEC) and prevent the growth of pathogenic organisms (Ronnqvist et al. 2006). Once the balance of the local microbiota is broken, it can predispose women to urogenital infections, such as bacterial vaginosis (BV) (Danielsson et al. 2011). Supplying selected lactobacilli might be a rational therapeutic strategy in restoring a healthy microbiota and preventing infections (Bolton et al. 2008; Reid et al. 2009). To meet this challenge, commercial EcoVag® vaginal capsules which consist of two strains (L. gasseri DSM 14869 and L. rhamnosus DSM 14870, 10⁸ CFU for each strain) isolated from healthy women vaginal epithelial cells have previously been developed and vaginal administration of EcoVag® capsules can eliminate the symptoms of BV in 90% of the patients (Stray-Pedersen et al. 2003, unpublished data). Supplement treatment with vaginal EcoVag® capsules can also effectively improve the therapeutic effect of antibiotic treatment of BV (Larsson et al. 2008; 2011; Pendharkar et al. 2015). In spite of the wealth of clinical data showing the health benefits of EcoVag® strains in humans, there is still a lack of understanding of the cell surface factors or molecular mechanisms underlying their probiotic activities.

Studies of gastrointestinal lactobacilli suggest that health-promoting effects of probiotics could be related to their capacity to adhere to intestinal epithelial cells and/or mucus, as it can promote colonization, pathogen exclusion and interactions with host (Lebeer et al. 2008; 2010). Thus a high adhesive ability to intestinal surface is considered to be a major distinguishing feature for the selection of probiotic strains. Bacterial adherence to host epithelial surface is often mediated by the cell surface components, including sortase-dependent proteins (SDPs) and other cell surface molecules, such as exopolysaccharides (EPS), lipoteichoic acid and S-layer proteins (Lebeer et al. 2008) and extracellular appendages, such as pili, fimbriae and flagella (Juge, 2012).

EPS contribute significantly to lactobacilli-host interactions, especially with intestinal mucosa and epithelial cells, thus contributing to the strain-specific probiotic characteristics (Lebeer et al., 2008). Bacterial polysaccharides vary in sugar composition, position of branches and modifications, contributing to the wide diversity of surface structures (Ruas-Madiedo et al. 2002). EPS have been reported to be involved in aggregation, biofilm formation, adhesive properties and immunomodulation by probiotic strains (Lebeer et al. 2009; Dertli et al. 2015; Lee et al. 2016; Živkoviĉ et al. 2016).

SDPs are an important group of cell surface proteins in Gram positive bacteria, which are best characterized in lactobacilli and suggested to play a key role in bacterial adhesion (Boekhorst et al. 2005). These SDPs share a common structure including a YSIRK signal peptide that promotes secretion (Bae & Schneewind, 2003), a C-terminal LPxTG anchoring motif, followed by a transmembrane helix and a positively charged tail (Lebeer et al. 2008; Jensen et al. 2014). After the surface protein precursor is transferred to the plasma membrane, it will be cleaved and covalently anchored to the cell wall by sortase A (Marraffini et al. 2006). Different SDPs have been identified in lactobacilli, including the mucus-binding pilin SpaC in L. rhamnosus GG (Kankainen et al. 2009), Lactobacillus epithelium adhesion (LEA) in L. crispatus ST1 (Edelman et al. 2012), mucus binding protein A (CmbA) in L. reuteri ATCC PTA6475 (Jensen et al. 2014) and mannose-specific adhesin Msl in L. plantarum CMPG5300 (Malik et al. 2016). However, until now, very few SDPs have been identified in human vaginal lactobacilli.

In previous studies, the inventors have found that the EcoVag® strains shown a highly efficient adhesion ability to vaginal epithelial cells. We have also performed complete genome sequencing and characterization of the two strains and found several genes potentially involved in bacterial probiotic activity (Marcotte et al. 2017). In particular, production of a thick (40 nm) EPS layer and a putative new adhesin containing three rib/alpha-like repeats was found in L. gasseri DSM 14869 (Marcotte et al. 2017). In this study, the inventors aimed to characterize the L. gasseri DSM 14869 surface molecules that mediate adhesion to the human vaginal mucosa, including the EPS, a protein (N506_1778) with mucus-binding like domain, and a putative novel protein (N506_1709) with rib/alpha-like repeat domains. The results suggest that the genes encoding EPS, N506_1778 and N506_1709 contribute to the ability of L. gasseri DSM 14869 to adhere to vaginal epithelial cells in vitro.

### Results

Identification of a putative EPS gene cluster in L. gasseri DSM 14869. The genome of L. gasseri DSM 14869 (Marcotte et al. 2017) harbors a putative EPS cluster composed of 16 genes (N506_0396 to N506_0411) (FIG. 7A) sharing a high degree of similarity to L. gasseri ATCC 33323 (Azcarate-Peril et al. 2008). These genes are predicted to be involved in EPS biosynthesis (FIG. 7A), including those encoding glycosyltransferases and proteins involved in polymerization, export, and chain length determination (Lebeer et al. 2009). Based on a BlastP analysis, the N506_0400 gene encodes a putative priming glycosyltransferases protein, sharing 91% amino acid homology with priming glycosyltransferase epsE in L. johnsonii F19785 (Horn et al. 2013). Priming glycosyltransferase has been demonstrated to be a necessary control point of EPS biosynthesis (Horn et al. 2013) and we thus hypothesized that the deletion of the putative priming glycosyltransferase gene (N506_0400) could affect L. gasseri DSM 14869 EPS production.

Deletion of the N506_0400 gene influences the total level of EPS. TEM pictures clearly showed that the N506_0400 gene deletion mutant strain (ΔEPS) produce significantly (p<0.001) less EPS layer around the cell surface compared to the wild type (WT) strain (FIG. 8). While the reintroduction of the functional N506_0400 gene into the mutant strain completely restored the thickness of EPS layer to the WT levels.

Deletion of the N506_0400 gene results in increased auto-aggregation and biofilm formation. When the N506_0400 mutant strain L. gasseri DSM 14869-ΔN506_0400 (ΔEPS) was grown in liquid medium, no significant difference in the growth rate was observed as compared to WT L. gasseri DSM 14869 (FIG. 14).

However, the mutant showed a cell sediment and a very clear upper solution while the WT strain showed a relatively homogenous suspension (FIG. 9A). The complement strain restored the phenotype to WT levels, displaying a homogenous suspension (FIG. 9A). The mutant strain L. gasseri DSM 14869-ΔN506_0400 also displayed a significant (p<0.001) increase of auto-aggregation ability, to 216% of that of the WT. The auto-aggregation was restored to 136% in the complemented strain (FIG. 9B). In addition, biofilm formation by the mutant strain was increased by 15-fold (p<0.001) compared to that by the WT as evaluated by the microtiter biofilm assay (FIG. 9C). Complementation partially restored biofilm formation, with an 8-fold increase compared to WT (FIG. 9C).

Impact of EPS on adhesion of L. gasseri DSM 14869 to different epithelial cells. Previous studies have shown that EPS from intestinal lactobacilli is involved in adhesion to intestinal epithelial cells (Lebeer et al. 2009; Lee et al. 2016; Živković et al. 2016). In this study, we investigated the role of EPS in the adhesive capacity of the vaginal strain L. gasseri DSM 14869. As observed in FIG. 10A, the EPS mutant L. gasseri DSM 14869-ΔN506_0400 showed a significant 20-fold increase (p<0.001) in adhesion to the colon carcinoma cell line Caco-2 compared to the WT strain. In addition, the EPS mutant showed also a significant roughly 14-fold increase (p<0.001) in adhesion to cervical cancer cell line HeLa (FIG. 10B). The mutant strain complemented with pNZe-N506_0400 gene showed restoration of the adhesive capacity to Caco-2 and HeLa cells nearly to WT level (FIG. 10A, B). We further investigated whether the EPS is also involved in the adhesion to vaginal epithelial cells. Interestingly, the EPS mutant strain showed a slight reduction (˜j 30%, (p<0.01) in adhesion to the vaginal epithelial cells (FIG. 100), while the complemented strain (14869-ΔN506_0400/pNZe-N506_0400) showed partial restoration of the adhesion (FIG. 100). This suggests that EPS in L. gasseri DSM 14869 might mediate tissue tropism specifically towards vaginal epithelial cells.

Sequence analysis of putative adhesion proteins in L. gasseri DSM 14869. Surface proteins are very important molecules involved in bacterial adhesion to mucus and epithelial cells. In the genome of L. gasseri DSM 14869, 22 predicated ORFs with putative adhesion-related domains have previously been identified (Marcotte et al. 2017). However, only two ORFs (N506_1778 and N506_1709) encode both a YSIRK-type signal peptide (PF04650) and a LP_{X}TG anchor motif (PF00746), which would result in a protein secreted and covalently anchor to the cell wall (FIG. 7B, C). Therefore, these two proteins were classified as SDPs and are both potentially involved in the adhesion of L. gasseri DSM 14869 to epithelial cells.

The N506_1778 gene in L. gasseri DSM 14869 is 5.055 kb long and encodes a protein of 1684 amino acid residues with a predicted molecular weight of 186.7 kD. The N-terminus is a YSIRK-type signal peptide and the C-terminus contains a LPQTG (LP_{X}TG-like cell wall anchor) motif which belongs to the gram-positive LP_{X}TG anchoring superfamily. The N506_1778 protein also contains 2 non-identical repeats (amino acid 986-1092 and 1384-1490), showing 66% amino acid identity (FIG. 7B). The two repeats show low homology with MucBP (mucin binding protein) (PF06458) domain with a 35%-39% aa identity with the Mub-RV repeat of the mucus-binding protein (MUB) from L. reuteri ATCC 53608 (Etzold et al. 2014), a 31-37% aa identity with Mub1 repeat of MUB from L. reuteri 1063 (MacKenzie et al. 2009) and a 36%-42% aa identity with MucBP domain (fragment 187-294) of protein LBA1460 from L. acidophilus NCFM. Many MUB homologues and MucBP domain-containing proteins have been identified in lactobacilli naturally located in intestinal niches and have been suggested to play an important role in the adherence of probiotic strains to the intestinal mucin (Ossowski et al. 2011; Etzold et al. 2014; Jensen et al. 2014) and epithelial cells (Call and Klaenhammer, 2013; Jensen et al. 2014). In this study, we found that N506_1778 and its homologues were also present in lactobacilli naturally located in vaginal niches. We subsequently compared the homologues of protein N506_1778 in all 10 known L. gasseri genomes (7 strains from the human vaginal tract) (table 3). Interestingly, N506_1778 homologues were found to be contained by all the known L. gasseri genome, with the aa identity 79-98% (table 3). The high similarities with other homologous proteins in L. gasseri species suggests that N506_1778 may play essential roles for this species to adapt to different host niches. Thus, double-crossover recombination was used to knock out the gene N506_1778 and the mutant was evaluated for its adherent capacity to different epithelial cells.

TABLE 3
Homologies of the N506_1778 with other genes in L. gasseri

| | | | Amino | |
|---|---|---|---|---|
| | | | acid | Function or |
| | | | identity | characteri- |
| Strain | Origin | Homologous gene | (%) | zation |
| L. gasseri | Human | LGAS_1655 | 1387/1420 | Signal peptide |
| ATCC33323 | intestine | (WP_003646778.1) | (98) | |
| L. gasseri | Human | A131_RS08225 | 1387/1420 | Signal peptide |
| CECT 5714 | milk | (WP_003646778.1) | (98) | |
| L gasseri K7 | Infant | LK7_RS06720 | 1362/1696 | Signal peptide |
| | feces | (NZ_KL402719) | (80) | |
| L. gasseri | Human | HMPREF9209_222 | 1482/1535 | LPXTG-motif |
| 224-1 | vaginal | 4 (EFB61564.1) | (97) | cell |
| | tract | | | wall anchor |
| | | | | domain protein |
| L. gasseri | Human | M497_RS0110705 | 1387/1420 | Signal peptide |
| 2016 | vaginal | (WP_003646778.1) | (98) | |
| | tract | | | |
| L. gasseri | Human | HMPREF0890_136 | 796/1010 | Gram-positive |
| 202-4 | vaginal | 3 (EEQ26348.1) | (79) | signal peptide |
| | tract | | | protein, |
| | | | | YSIRK family |
| L. gasseri | Human | HMPREF0514_119 | 1359/1706 | Signal peptide |
| JV-V03 | vaginal | 12 | (80) | |
| | tract | (WP_003650025.1) | | |
| L. gasseri | Human | LBGG_01576 | 1387/1420 | Gram-positive |
| MV-22 | vaginal | (EFQ45690.1) | (98) | signal peptide |
| | tract | | | protein, |
| | | | | YSIRK family |
| L. gasseri | Human | HMPREF0516_015 | 1387/1420 | Putative |
| SJ-9E-US | vaginal | 90 (KFL94804.1) | (98) | YSIRK type |
| | tract | | | signal peptide |
| L. gasseri | Human | HMPREF5175_017 | 1482/1535 | Putative |
| SV-16A-US | vaginal | 79 (KFL96562) | (97) | LPXTG- |
| | tract | | | motif cell wall |
| | | | | anchor domain |
| | | | | protein |

The N506_1709 gene in L. gasseri DSM 14869 consists of a 4.371 kb sequence encoding a large surface protein of 1456 amino acids with a predicted molecular weight of 158.9 kD. This protein includes a YSIRK signal peptide, a N-terminal region (amino acids 42 to 1233), an internal repeat region (amino acids 892 to 1372) harboring three repeats (the first one is partial) which shows similarity to rib/alpha-like repeats domain (PF08428) in Pfam analysis, and a LPQTG anchoring motif in C-terminus (FIG. 7C). After BLASTP analysis, the inventors found that N506_1709 has high sequence identity (99%) with a hypothetical protein (LJCM1025_14810) from L. gasseri LJCM1025 but less than 10% identity with other proteins in the databank. The last 600 aa, containing the rib/alpha-like repeats region, shows 34%-48% aa identity with surface proteins from L. johnsonii and L. gasseri (FIG. 7C). The rib/alpha-like repeats domain was also found in several cell surface proteins of lactobacilli and it was suggested that proteins with this domain may promote bacterial adhesion to stratified squamous epithelial cells (Edelman et al. 2012; Stalhammar-Carlemalm et al. 1999). Since the protein sequence features of N506_1709 suggest that it might be a new putative adhesion protein promoting the binding of L. gasseri DSM14869 to vaginal epithelial cells, the encoding gene was also deleted by double-crossover recombination.

Construction of N506_1778 and N506_1709 knockout mutants. As shown in FIGS. 11A, B, in N506_1778 and N506_1709 mutants, no mRNA of genes N506_1778 and N506_1709 were expressed, while the mRNA expression levels of the corresponding genes in complementary strains were restored to wild type. The results indicated that genes N506_1778 and N506_1709 were successfully deleted from the genome of L. gasseri DSM14869.

N506_1778 Mediated Binding of L. gasseri DSM 14869 to Caco-2, HeLa and Human Vaginal Cells.

The growth rate of the N506_1778 mutant strain was not altered under the growth conditions used in the study (FIG. 14). The effects of N506_1778 mutation were determined by evaluating the ability of the mutant to adhere to Caco-2, HeLa and vaginal epithelial cells in vitro. As shown in FIG. 12A-C, the mutation of N506_1778 resulted in a significant reduction in adhesion to Caco-2 (42%, p<0.01), HeLa (32%, p<0.001) and human vaginal cells (32%, p<0.01) as compared to wild-type strain. The complementation with pNZe-N506_1778 restored the wild-type level adhesion capacity to vaginal cells (FIG. 12C). These results indicate that N506_1778 is an important cell surface protein involved in adhesion of L. gasseri DSM 14869 to different host epithelial cells.

N506_1709 mediated binding of L. gasseri DSM 14869 to human vaginal cells but not to Caco-2 and HeLa cells. In order to determine the contribution of N506_1709 to bacterial adhesion, a N506_1709 knockout mutant was constructed. The mutant strain showed the same growth rate with wild-type (FIG. 14). The mutant was first evaluated for its adhesion to columnar epithelial cell lines Caco-2 and HeLa. As shown in FIG. 6A, B, the N506_1709 mutant strain L. gasseri DSM 14869-ΔN506_1709 did not show a significant difference for adhesion to Caco-2 and HeLa cells as compared to the wild-type strain. Since N506_1709 includes a rib/alpha-like repeat domain, which has been suggested to be involved in binding to stratified squamous epithelial cells (Edelman et al. 2012), the inventors subsequently investigated if N506_1709 plays a role in adhesion to human vaginal cells, a type of stratified squamous epithelial cells. As displayed in FIG. 12C, the N506_1709 mutant showed a significant ca. 42% reduction (p<0.001) in adhesive ability to human vaginal cells as compared to wild-type. To confirm the relation of the genotype-phenotype for the N506_1709 gene, the mutant strain was subsequently complemented by re-introducing the N506_1709 gene. The complemented strain L. gasseri DSM 14869-ΔN506_1709/pNZ8048-N506_1709 showed partial restoration of the adhesive levels (FIG. 12C), suggesting that N506_1709 mediates tissue-specific adhesion towards vaginal stratified squamous epithelial cells.

Overexpression of N506_1709 in Lactococcus lactis NZ9000 increases adhesion to vaginal epithelial cells. To confirm tissue specific adhesion by N506_1709, the protein N506_1709 was overexpressed in L. lactis NZ9000 using the pNZ8048 vector and its nisin-inducible expression system. L. lactis NZ9000 transformed with the empty plasmid pNZ8048 was used as a control. After nisin induction of N506_1709 expression in L. lactis NZ9000, the overexpressed strain showed a 5000-fold increase of the mRNA transcription compared to control (FIG. 13A). In addition, the adhesion ability to vaginal cells for the overexpressed strain also showed a 2.8-fold increase (p<0.001) (FIG. 13B), confirming that N506_1709 plays a role in the adhesive capacity of L. gasseri DSM 14869 to bind to human vaginal epithelium.

### Discussion

L. gasseri is one of the major species isolated in the vaginal microbiota (Pendharkar et al. 2013; Ravel et al. 2011) and several health benefits have been reported in L. gasseri strains (Marcotte et al. 2017; Parolin et al. 2015). However, the molecular mechanisms underlying the health-promoting effects, such as the adhesion factors that allow optimal adhesion of lactobacilli to this niche, are in general not yet well understood. In this study, we genetically identified and functionally analyzed three genes that may be involved in adhesion of the probiotic strain L. gasseri DSM 14869 to vaginal epithelial cells.

First, the function of the identified EPS gene cluster in L. gasseri DSM 14869 was evaluated by mutation of the N506_0400 gene, which encodes the putative priming glycosyltransferase. According to the literature, the gene encoding priming glycosyltransferase is highly conserved (Jolly and Stingele, 2001) and priming glycosyltransferase plays an essential role in the first step of EPS biosynthesis by transferring the first sugar to the UndP-lipid carrier (Lebeer et al. 2009). Mutation of the genes encoding priming glycosyltransferase of L. johnsonii, L. rhamnosus or L. paracasei abolished or reduced heteropolysaccharide production (Lebeer et al. 2009; Horn et al. 2012; Živković et al. 2016). In this study, the increased auto-aggregation ability and reduced expression of surface-associated polysaccharide of the N506_0400 mutant strain versus the wild-type also indicate a crucial role for the priming galactosyltransferase N506_0400 in the biosynthesis of EPS of L. gasseri DSM 14869. At the same time, the EPS mutant of L. gasseri DSM 14869 showed a significant increase of biofilm formation when grown in MRS medium, while the complemented strain restored part of the biofilm formation capacity to wild-type (FIG. 9C). Biofilm formation is considered to be one of the important surface properties of probiotics involved in their beneficial effects on the host (Younes et al. 2012; Jones & Versalovic, 2009). It is suggested that the capacity for biofilm formation may be related to sortaseA dependent proteins (SDPs). For example, the EPS mutant of L. rhamnosus GG showed a substantial increase in biofilm formation which was speculated to be due to the exposure of more cell surface adhesins after removing the EPS (Lebeer et al. 2009; Lebeer et al. 2012). Malik et al. (2013) also reported that biofilm formation in the vaginal L. plantarum CMPG 5300 strain could be due to SDPs since a srtA mutant of this strain lost its biofilm-formation capacity. Therefore, in this study, the increased biofilm-formation ability of the EPS mutant strain could also be by virtue of the exposed SDPs.

Furthermore, the role of EPS on adhesion was studied using Caco-2, HeLa and vaginal epithelial cells. The EPS mutant showed a significant increase adhesive capacity to colon carcinoma cells Caco-2 and HeLa cervical carcinoma cells but a slightly reduced adhesion to vaginal epithelial cells. The increased adhesion to Caco-2 and HeLa could potentially be attributed to a better exposure of adhesins because of the absence of EPS. These data are consistent with studies of L. rhamnosus GG showing that deprivation of galactose-rich EPS increased adhesion to Caco-2 cells possibly because of an enhanced exposure of adhesins (Lebeer et al. 2009) and additional studies about characterization of the specific adhesins of L. rhamnosus GG such as pili substantiate this “shielding hypothesis” (Lebeer et al. 2012). Similar results were also reported in the L. johnsonii NCC533 (Denou et al. 2008), L. rhamnosus E/N (Polak-Berecka et al. 2014) and L. plantarum Lp90 strains (Lee et al. 2016). However, the adhesive capacity of the EPS mutant to vaginal epithelial cells was slightly decreased (30%), suggesting that EPS may possibly promote adhesion of L. gasseri DSM 14869 to vaginal epithelial cells. The role of EPS on adhesion is strain-specific and could probably be related to differences in structural characteristics of the EPS, as well as to the cell surface characteristics of the strain. For example, the mutation of cps clusters in the L. plantarum strains WCFS1 and SF2A35B has no significant influence on adhesion to Caco-2 cells (Lee et al. 2016). Živković et al. (2016) reported that the presence of the EPS-SJ P2 increases adhesion to Caco-2 cells which may be attributed to the molecular structure of EPS-SJ P2 matrix. Hence, specific increase in L. gasseri DSM 14869 adhesion to vaginal cells by EPS may be due to a different molecular structure of EPS or different receptors in vaginal cells compared to Caco-2 and HeLa cells. How the EPS promotes bacterial adhesion to vaginal cells need to be further studied. To the best of our knowledge, this is the first report demonstrating that the EPS of vaginal lactobacilli might provide tissue tropism adhesion. This will help to better understand its specific contribution in probiotics-host interactions and its role in adaptation to this vaginal niche.

Increased adhesion to the intestinal mucosal layer by some lactobacilli has been suggested to be mediated by cell surface proteins with a mucus-binding capacity (Kankainen et al. 2009; Rojas et al. 2002; Ossowski et al. 2011; Jensen et al. 2014). The N506_1778 protein, harboring 2 repeats with homology to MucBP domain, was the only predicated cell wall anchored protein that includes MucBP-like domain in L. gasseri DSM 14869. Our results showed that the N506_1778 protein could promote adherence of L. gasseri DSM 14869 to Caco-2, HeLa and human vaginal epithelial cells. N506_1778 homologues were found in all the known L. gasseri strains isolated from different niches (Table 3), suggesting that N506_1778 is an important cell surface protein for L. gasseri species to adapt to different host niches. This is the first time that a protein with MucBP-like domain has been reported to also be involved in adhesion to vaginal epithelial cells.

N506_1709 is another important cell surface protein mediating adhesion of L. gasseri DSM 14869 to vaginal mucosal cells. It is a newly described sortase-dependent adhesin that shows specific binding to stratified squamous epithelial cells. Interestingly, N506_1709 differs from the previously characterized Lactobacillus adhesins, such as Lsp, Mub and mucus-binding factor (MBF) (Walter et al. 2005; Buck et al. 2005; von Ossowski et al. 2011), as it contains no MuBP domains but instead harbors three repeated regions with homology to Rib/a-like repeats. Rib and alpha proteins were first identified in Streptococcus and were suggested to be involved in pathogen adhesion and biofilm formation (Michel et al. 1992; Wästfelt et al. 1996; Stâlhammar-Carlemalm et al. 1999). Later, proteins showing homology with Rib/a-like repeats were also reported in vaginal lactobacilli, such as protein Rip in L. fermentum and LEA in L. crispatus (Turner et al. 2003; Edelman et al. 2012). These proteins with Rib/a-like repeats domain in lactobacilli were suggested to mediate binding to the stratified squamous epithelial lining of the host (Edelman et al. 2012; Turner et al. 2003). In this study, the N506_1709 mutant showed a significantly reduced adhesive capacity to vaginal epithelial cells (stratified squamous epithelial cells), but not to colon carcinoma cells and cervical carcinoma cells (columnar epithelial cells). This suggests that the N506_1709 protein provides tissue tropism to L. gasseri DSM 14869, likely determined by the presence of different receptors on the cell membrane of vaginal epithelial cells. The over-expression of N506_1709 in L. lactis significantly improved L. lactis adhesion to vaginal epithelial cells, further confirming the adhesive capacity of N506_1709 to vaginal epithelial cells. Taken together, N506_1709 is an important surface protein mediating the adherence of L. gasseri to human vaginal epithelium, which could promote the bacterial colonization in the host and may be of ecological importance.

In conclusion, the current report identifies and functionally analyzes three cell surface molecules including EPS, N506_1778 and N506_1709 as important adhesion factors of L. gasseri DSM 14869 involved in vaginal adhesion. To our knowledge, this is the first report that demonstrates the role of EPS in adherence of a vaginal Lactobacillus strain and that a protein with MucBP-like domains could also be involved in vaginal epithelium adhesion. In addition, N506_1709 might be a novel adhesin specifically mediating bacterial binding to stratified squamous epithelial cells and was annotated as Lactobacillus vaginal epithelium adhesin (LVEA). The results provide important new information on the molecular mechanisms of Lactobacillus adhesion and tissue tropism to mucosal surfaces of various hosts, and could help us to screen for better probiotic candidates in the future. Further studies are still needed to address the specific receptors for EPS and LVEA on vaginal epithelial cells as well as the functional domains of LVEA.

### Materials and Methods

Bacterial strains, plasmids, and growth conditions. The bacterial strains and plasmids used in this study are listed in Table 4.

TABLE 4
Bacterial strains and plasmids used in this study

| | | Source or |
|---|---|---|
| Strains and plasmids | Relevant characteristic(s)^{a} | reference |
| Strains | | |
| E.coli VE7108 | Containing the repA plasmid | Mora et al. |
| | gene (not thermosensitive), | (2004) |
| | Km^{r} | |
| L. gasseri DSM 14869 | Parent strain | BifodanA/S, |
| | | Denmark |
| 14869-ΔN506_0400 | 14869 lacking the gene | This study |
| | N506_0400 | |
| 14869-ΔN506_1778 | 14869 lacking the gene | This study |
| | N506_1788 | |
| 14869-ΔN506_1709 | 14869 lacking the gene | This study |
| | N506_1709 | |
| 14869-ΔN506_0400/pNZe- | 14869-AN506_0400 | This study |
| N506_0400 | harboring plasmid pNZe- | |
| | N506_0400 | |
| 14869-ΔN506_1778/pNZe- | 14869-ΔN506_1778 | This study |
| N506_1778 | harboring pNZe-N506_1778 | |
| 14869-ΔN506_1709/ | 14869-ΔN506_1709 | This study |
| pNZ8048-N506_1709 | harboring pNZ8048- | |
| | N506 _1709 | |
| Lactococcus lactis NZ9000 | MG1363 pepN::nisRK | Kuipers et al. |
| | | (1998) |
| L. lactis NZ9000/pNZ8048 | L. lactis NZ9000 harboring | This study |
| | empty vector pNZ8048 | |
| L. lactis NZ9000/pNZ8048- | L. lactis NZ9000 harboring | This study |
| N506_1709Re | pNZ8048-N506_1709Re | |
| Plasmids | | |
| pINTZrec | Replicable plasmid mediating | (unpublished |
| | homologous recombination, | data) |
| | Cm^{r} | |
| pINTZrec-N506_0400 | pINTZrec derivative | This study |
| | containing homologous | |
| | regions up- and downstream | |
| | of N506_0400; Cm^{r} | |
| pINTZrec-N506_1778 | pINTZrec derivative | This study |
| | containing homologous | |
| | regions up- and downstream | |
| | of N506_1778; Cm^{r} | |
| pINTZrec-N506_1709 | pINTZrec derivative | This study |
| | containing homologous | |
| | regions up- and downstream | |
| | of N506_1709; Cm^{r} | |
| pNZ8048 | E. coli-lactic acid bacteria | de Ruyter et al. |
| | shuttle cloning vector; Cm^{r} | (1996) |
| pNZe-Rec | pNZ8048 derivative, Em^{r} | Laboratory |
| | | stock |
| pNZe-N506_0400 | pNZe-Rec derivative | This study |
| | containing N506_0400; Em^{r} | |
| pNZe-N506_1778 | pNZe-N506_0400 derivative | This study |
| | containing N506_1778; Em^{r} | |
| pNZ8048-N506_1709 | pNZ8048 derivative | This study |
| | containing N506_1709 and | |
| | its promoter; Cm^{r} | |
| pNZ8048-N506_1709Re | pNZ8048 derivative | This study |
| | containing N506_1709, Cm^{r} | |

^{a}Km^{r}, kanamycin resistant; Cm^{r}, chloramphenicol resistant; Em^{r}, erythromycin resistant.

Escherichia coli VE7108 (Mora et al. 2004) strain was incubated in Luria broth supplemented with 25 μg ml⁻¹ of kanamycin at 37° C. with shaking. Lactobacillus strains were statically grown in MRS broth at 37° C. under anaerobic conditions. When required, the following antibiotics were added: 10 μg ml⁻¹ chloramphenicol (Cm) and 300 μg ml⁻¹ erythromycin (Em) for E. coli; 10 μg ml⁻¹ Cm and 5 μg ml⁻¹ Em for Lactobacillus transformants.

DNA manipulations. Standard DNA protocols were used for DNA manipulations in E. coli (Sambrook et al. 1989). Plasmid DNA of E. coli was extracted using QIAGEN Miniprep Spin Kit (Qiagen, Hilden, Germany). L. gasseri DNA was isolated using QIAamp DNA Stool Mini kit (Qiagen, Hilden, Germany). Primers (Table 5) were synthesized by Eurofins Genomics (Ebersberg, Germany). Phusion High-Fidelity DNA polymerase (Finnzymes/Thermo Fisher Scientific, Espoo, Finland) was used for amplification of L. gasseri genomic DNA and GoTaq DNA polymerase (Promega, Fitchburg, Wis., USA) was used for colony PCR. The PCR products were purified by QIAquick Gel Extraction Kit (Qiagen, Hilden, Germany). The restriction endonucleases were supplied by Thermo Scientific and T4 DNA ligase was from Invitrogen (Carlsbad, Calif.). All of the procedures were conducted according to the manufacturer's instructions. Plasmids were transformed into L. gasseri DSM 14869 by electroporation as described previously (De Keersmaecker et al. 2006).

TABLE 5
List of primers used in this study

| | | |
|---|---|---|
| | | Restriction |
| Primer name | Sequence (5′-3′)^{a} | site |

Primers for gene knock out

| | | |
|---|---|---|
| 400up5tream-F | ACACAGAGCTCAGCTGGTGAAATGGATGGC | Sac I |
| | (SEQ ID NO: 19) | |
| 400upstream-R | TTAACTTTTCTCCTTAAAATAACCAAAATCTT | |
| | TTCTT (SEQ ID NO: 20) | |
| 400downstream-F | TTGGTTATTTTAAGGAGAAAAGTTAAAAGTA | |
| | TG (SEQ ID NO: 21) | |
| 400downstream-R | ACCAAGCTAGCTGTATCACTTTTGGTATTGA | Nhe I |
| | (SEQ ID NO: 22) | |
| Seq1-F | ATTGGGTTCTAACAGAATGCGT (SEQ ID NO: | |
| | 23) | |
| INTZ-R | TTCTCCCCCTAATAATTCTGCAG (SEQ ID NO: | |
| | 24) | |
| INTZ-F | GGTTTTTATATTACAGCTCCAAG (SEQ ID NO: | |
| | 25) | |
| Seq1-R | AATCCTGAATCACTTTCGGTTT (SEQ ID NO: | |
| | 26) | |
| 1778upstream-F | CAACGGAGCTCCGAAACTAATGGCATCAAT | Sac I |
| | (SEQ ID NO: 27) | |
| 1778upstream-R | GTTTTTGGTGCAGAGAAATTTATATATAAAA | |
| | TAACGATTTTGT (SEQ ID NO: 28) | |
| 1778downstream- | TTGTATAGGAGAAACAAGGTTTCTTGATAAT | |
| F | AAATAACATTAATAGC (SEQ ID NO: 29) | |
| 1778downstream- | CTTTAAGGATCCAAAAGAAGAAGCTAGAAA | Bam HI |
| R | GGCTT (SEQ ID NO: 30) | |
| Seq2-F | CACATAATACCAGCAGTCAACGAA (SEQ ID | |
| | NO: 31) | |
| Seq2-R | GTGCTCCAAGTAGTATCATAGCGAT (SEQ ID | |
| | NO: 32) | |
| 1709upstream-F | TTCCTGGAGCTCAAACTTTATTTTGTTCTGC | Sac I |
| | CAA (SEQ ID NO: 33) | |
| 1709upstream-R | ATGTTATTTATTATCAAGAAACCTTGTTTCTC | |
| | CTATACAATGT (SEQ ID NO: 34) | |
| 1709downstream- | TTGTATAGGAGAAACAAGGTTTCTTGATAAT | |
| F | AAATAACATTAATAGC (SEQ ID NO: 35) | |
| 1709downstream- | CTTTAAGGATCCAAAAGAAGAAGCTAGAAA | Bam HI |
| R | GGCTT (SEQ ID NO: 36) | |
| Seq3-F | GGCTAAAAACAGACTCCATCAATC (SEQ ID | |
| | NO: 37) | |
| Seq3-R | AGCCCGTTCTTTCTTATAACTTTAA (SEQ ID | |
| | NO: 38) | |

Primers for complementation

| | | |
|---|---|---|
| EPS-Promoter-F | AATTAGGTACCACACTGTAAAAATAAATAAG | Kpn I |
| | ATCCT (SEQ ID NO: 39) | |
| EPS-Promoter-R | TTAACCTCTTGTGCCATCATTTTATTCCTCTT | |
| | TTATTTTT (SEQ ID NO: 40) | |
| N506_0400-F | AAAAATAAAAGAGGAATAAAATGATGGCACA | |
| | AGAGGTTAA (SEQ ID NO: 41) | |
| N506_0400-R | CTTTTAAGCTTAATACGCACTATTTGGATGA | Hin dIII |
| | AT (SEQ ID NO: 42) | |
| N506_1778-F | TACATCGGTACCAAAATATGCGGTATGTATT | Kpn I |
| | TATCG (SEQ ID NO: 43) | |
| N506_1778-R | GGTGCAGGATCCTTAATTCTTTTTCTTTCGT | BaM HI |
| | TTAAGTT (SEQ ID NO 44) | |
| N506_1709-F1 | AAGACAGATCTCGTAATTAAATTGATCAAGT | Bgl II |
| | ACATTAT (SEQ ID NO: 45) | |
| N506_1709-R1 | ACAAAGGTACCACCAGATTCCAT (SEQ ID | Kpn I |
| | NO: 46) | |
| N506_1709-F2 | CTGGTGGTACCTTTGTTTCAAAAGT (SEQ ID | Kpn I |
| | NO: 47) | |
| N506_1709-R2 | ATTATGAGCTCTTATCTAATTCGGTGTTTTC | Sac I |
| | TTCTACTT (SEQ ID NO: 48) | |

Primers for overexpression of
N506_1709 in L. lactis

| | | |
|---|---|---|
| N506_1709Re-F | GAGAAACCATGGATGCTATCTAAAAATAATT | Nco I |
| | TTCATG (SEQ ID NO: 49) | |
| N506_1709 Re-R | TTAATGAGCTCTTACGCTTCCGGTTCTCTAA | Sac I |
| | TTCGGTGTTTTCTTCTACTT (SEQ ID NO: 50) | |

Primers for qRT-PCR

| | |
|---|---|
| L. gasseri 16S-F | ACCCTTGTCATTAGTTGCCATCA (SEQ ID |
| | NO: 51) |
| L. gasseri 16S-R | GCTTCTCGTTGTACCGTCCATT (SEQ ID NO: |
| | 52) |
| 1778-F | AGACCTTAGAGAGCAAGTCATTATCG (SEQ |
| | ID NO: 53) |
| 1778-R | TTGGTTATTAGGAAGTTCGTCGTT (SEQ ID |
| | NO: 54) |
| 1709-F | ATTGGAACGATTTGAAGAGCG (SEQ ID NO: |
| | 55) |
| 1709-R | GAATCAGTAGTGTGGGAACCGAC (SEQ ID |
| | NO: 56) |
| L. lactis 16S-F | TCGTGTCGTGAGATGTTGGGT (SEQ ID NO: |
| | 57) |
| L. lactis 165-R | GTCATAAGGGGCATGATGATTTG (SEQ ID |
| | NO: 58) |

^{a}The restriction site is underlined in primer sequence.

Construction of EPS (N506_0400), N506_1778 and N506_1709 knock-out mutants by double homologous recombination. In this study, the replicable plasmid pINTZrec (unpublished data) (FIG. 15A) was used to mediate homologous recombination. This plasmid includes two six sites and a β-recombinase that specifically catalyzes the recombination between two six sites that flanked the antibiotic resistance gene and DNA to be integrated. The expression of β-recombinase is controlled by the sakacin-inducible promoter. For the deletion of N506_0400 gene from the genome of L. gasseri DSM 14869, about 1.0 kb up- and downstream fragments flanking the 5′ and 3′ ends of the N506_0400 gene were amplified by PCR using the primers 400upstream-F/R and 400downstream-F/R, respectively (Table 5). The generated amplicons were joined by overlap extension strategy using the primer pair 400upstream-F/downstream-R (Table 5). The resulting PCR products were digested with Sac I and Nhe I and ligated into similarly digested pINTZrec plasmid and transformed into electrocompetent E. coli VE7108 to obtain the final plasmid construct pINTZrec-N506_0400. Then pINTZrec-N506_0400 was electrotransformed into competent L. gasseri DSM 14869 cells which were prepared according to De Keersmaecker et al. (2006). For recombination-activated gene expression, the β-recombinase gene expression was induced with sakacin.

Briefly, the transformed L. gasseri strain was inoculated into MRS medium and when growth reached OD₆₀₀-0.5, 100 ng ml⁻¹ sakacin was added for overnight induction. Serial dilutions of induced culture were plated on MRS agar plates (with 10 μg ml⁻¹ Cm and 100 ng ml⁻¹ sakacin) and anaerobically grown for 48 h. To obtain the replicable plasmid already excised and single crossover integration, individual colonies with Cm gene and without repA gene were selected and further confirmed by PCR using seq1-F/INTZ-R and INTZ-F/seq1-R primers (table 5). The single crossover strain was grown in 3 ml MRS medium without antibiotics, and two subcultures per day were grown during 2 days. Subsequently, bacterial cultures were diluted and plated into MRS plates without antibiotics for 48 h to obtain single-colony isolates and then, these colonies were replica plated on MRS plates with 10 μg ml⁻¹ Cm. The non-antibiotic-resistant colonies were checked by specific primers seq1-F/seq1-R to obtain N506_0400 gene deletion strain, L. gasseri DSM 14869-ΔN506_0400.

For the deletion of N506_1778 and N506_1709 gene from the genome of L. gasseri DSM 14869, the same method for EPS knockout was used. Briefly, about 1.0 kb up- and downstream fragments flanking the 5′ and 3′ ends of the N506_1778 gene and N506_1709 gene were amplified by PCR using the primers 1778upstream-F/R and 1778downstream-F/R, and 1709upstream-F/R and 1709downstream-F/R, respectively. The generated amplicons were joined by overlap extension strategy using the primer pair 1778upstream-F/downstream-R and 1709 upstream-F/downstream-R (Table 5). The resulting PCR products were digested with Sac I and Bam HI and ligated into similarly digested pINTZrec plasmid and transformed into E. coli VE7108 to obtain the plasmid constructions pINTZrec-N506_1778 and pINTZrec-N506_1709. Plasmids pINTZrec-N506_1778 and pINTZrec-N506_1709 were electrotransformed into DSM 14869, respectively, and the transformed strains were induced by sakacin. Single crossover integrated colonies were screened by PCR using seq2-F/INTZ-R and INTZ-F/seq2-R primers for N506_1778 and seq3-F/INTZ-R and INTZ-F/seq3-R primers for N506_1709. Finally, double crossover integrated strains were checked by specific primers seq2-F/seq2-R to obtain N506_1778 gene deletion strain named L. gasseri DSM 14869-ΔN506_1778 and seq3-F/seq3-R to obtain N506_1709 gene deletion strain named L. gasseri DSM 14869-ΔN506_1709.

Complementation: plasmid construction and transformation. The plasmid pNZe-Rec was used as the starting material to achieve the plasmid required for the complementation of the gene N506_0400 in strain L. gasseri DSM 14869-ΔN506_0400. The gene N506_0400 does not have its own promoter; it shares the promoter of the EPS operon. Thus the promoter of EPS gene cluster was amplified using primers EPS-promoter-F/R (table 5) and N506_0400 gene was amplified with primers N506_0400-F/R. The promoter and N506_0400 gene were joined by overlap extension using the primer pair EPS-promoter-F/N506_0400-R and then digested with Kpn I and Hin dIII and ligated into similarly digested pNZe-Rec, resulting in pNZe-N506_0400. Plasmid pNZe-N506_0400 was electroporated into L. gasseri DSM 14869-ΔN506_0400 to yield an Em-sensitive strain, L. gasseri DSM 14869-ΔN506_0400/pNZe-N506_0400.

Plasmid pNZe-N506_0400 was used to complement the gene N506_1778 in strain L. gasseri DSM 14869-ΔN506_1778. The gene N506_1778 and its promoter were amplified by PCRs using the specific primers N506_1778-F/R. The PCR product was digested with Kpn I and Bam HI and ligated into pNZe-N506_0400 which was digested with Kpn I and Bgl II (Bam HI and Bgl II are isocaudarners), resulting in pNZe-N506_1778. Plasmid pNZe-N506_1778 was electroporated in L. gasseri DSM 14869-ΔN506_1778 to yield an Em-sensitive strain, L. gasseri DSM 14869-ΔN506_1778/pNZe-N506_1778.

Plasmid pNZ8048 was used to complement the gene N506_1709 in strain L. gasseri DSM 14869-ΔN506_1709. N506_1709 gene was ligated into pNZ8048 in two steps. First, the first part of gene N506_1709 (˜2 kb) and its promoter was amplified using primers N506_1709-F1/R1. The PCR product was digested with Bgl II and Kpn I and ligated into similarly digested pNZ8048, generating plasmid pNZ8048-N506_1709-1. Then the second part of gene N506_1709 (˜2.3 kb) was amplified using primers N506_1709-F2/R2 and digested with Kpn I and Sac I and ligated into similarly digested pNZ8048-N506_1709-1, resulting in pNZ8048-N506_1709. Plasmid pNZ8048-N506_1709 was electroporated into L. gasseri DSM 14869-ΔN506_1709 to yield a Cm-sensitive strain, L. gasseri DSM 14869-ΔN506_1709/pNZ8048-N506_1709.

Construction of overexpression constructs of N506_1709 in Lactococcus lactis NZ9000. For heterologous expression of N506_1709 in L. lactis, a nisin-inducible vector pNZ8048 was used. The N506_1709 gene from L. gasseri DSM14869 was amplified using primers N506_1709 Re-F/R (Table 5) and subsequently cloned into the pNZ8048 vector resulting in plasmids pNZ8048-N506_1709 Re. Competent L. lactis NZ9000 cells were transformed with plasmid pNZ8048-N506_1709 Re, resulting into strain L. lactis NZ9000/pNZ8048-N506_1709Re.

RNA extraction and Quantitative Real-Time PCR (qRT-PCR). Total RNA was extracted from 10⁹ bacteria grown in the logarithmic phase using the RNeasy Mini Kit (Qiagen). Reverse transcription was performed using QuantiTect Reverse Transcription Kit (Qiagen), containing 1 μg of total RNA as the template. qRT-PCR was carried out by using a SYBR Green assay kit (Qiagen). Specific primers (Table 5) were designed by Primer Premier 5 software and the internal gene 16S rRNA was used as reference. The relative gene expression was calculated by using the ΔΔC_{T} method (Schmittgen & Livak, 2008).

Analysis of auto-aggregation. Auto-aggregation analysis was performed as previously reported with some modifications (Leccese Terraf1 et al. 2014). Briefly, bacteria were grown overnight (˜16 h) in MRS. The cultures were centrifuged and washed twice with phosphate-buffered saline (PBS) (pH 7.2) and suspended in PBS to an OD₆₀₀ of 1.5. After incubation for 5 h at room temperature, the OD₆₀₀ of the upper suspension was measured. The auto-aggregation percentage was calculated by the expression as follows: auto-aggregation (%)=(1−(OD₆₀₀ 5 h/OD₆₀₀ 0 h))×100, where OD₆₀₀ 5 h represents the absorbance at the 5 h time point and OD₆₀₀ 0 h represents the absorbance at 0 h.

Biofilm formation assay. Biofilm formation was performed as described previously (Lebeer et al. 2007) with some modifications. Briefly, the biofilms were grown in MRS medium in 96-well polystyrene microplates at 37° C. for 72 h. Then the wells were washed three times with PBS and stained for 30 min with 0.1% crystal violet. Excess stain was rinsed with water and wells were air dried (1 h). The dye bound to the adherent cells was extracted with 200 μl 30% glacial acetic acid. The OD₅₇₀ of 135 μl of each well was measured. The experiments were repeated three times, each with eight replicates. Additionally, a sterile MRS medium was used as negative control.

Excess stain was rinsed with 200 II distilled water per well.

Transmission electron microscopy (TEM). Cells of L. gasseri DSM 14869, EPS mutant and complemented strains were grown overnight in MRS and the presence of EPS layer on the surface of L. gasseri strains was analysed by TEM as previously described (Alvarez et al. 2015). The relative thickness was expressed as a percentage relative to WT (set at 100%). Thickness was evaluated in 10 cells for each group and the mean±SD of thickness per cell was determined.

Assay of adhesion to Caco-2 and HeLa cells. Caco-2 and HeLa cells were routinely grown in DMEM medium supplemented with 10% fetal bovine serum (FBS), 100 IU ml⁻¹ penicillin G and 100 μg ml⁻¹ streptomycin. Adhesion assays were performed as previously described with some modifications (Lee et al. 2016). Briefly, cells were seeded in 24-well plates at a concentration of 10⁵ cells per well and cultured for 72 h until confluence. L. gasseri DSM 14869 was grown for 18 h and washed two times with PBS, and resuspended in antibiotic-free Dulbecco's modified Eagle's medium (DMEM) with a concentration of 10⁷ CFU ml⁻¹. 0.8 ml bacterial culture was added to the tissue culture wells and incubated for 2 h. The wells were washed 3 times with PBS to remove unadhered bacteria. Following washing, 0.2 ml trypsin-EDTA (Invitrogen) was added to the wells to detach the cells and then 0.6 ml antibiotic-free DMEM medium was added to stop the digestion of trypsin. Serial dilutions were prepared and plated onto MRS agar plates to count the number of adhered bacteria. The adhesion ratio was calculated as percent of bacteria adhering to Caco-2 or HeLa cells in relation to the total number of bacteria added in the wells. All adhesion experiments were performed in triplicate and repeated three times.

Assay of adhesion to vaginal epithelial cells (VEC). The protocol was approved by the Stockholm ethics committee (Regionala etikprövningsnämnden i Stockholm) (Permit Number: 2018/1090/31). Informed consent was obtained from participants before the start of the study. VEC cells were collected from 4 healthy volunteer donors by gently scraping the vaginal mucosal surface with a sterile cotton swab and suspended in 10 ml DMEM medium. The cells were washed three times with 10 ml DMEM and centrifuged at 800×g for 5 min to remove indigenous bacteria. The cells were adjusted to 10⁵ cells ml⁻¹ in DMEM by using a hemocytometer. L. gasseri DSM 14869 harvested from an 18 h culture, were washed twice with PBS (pH 7.4) and re-suspended in DMEM medium to get a final concentration of 5×10⁷CFU ml⁻¹. In the overexpression adhesion assays, the L. lactis strains were inoculated to OD₆₀₀˜0.5 and induced with 10 ng mL⁻¹ nisin (50 IU mL⁻¹, Sigma) for 1.5 h. Then the induced cultures were harvested as described above.

Equal volumes (400 μl) of vaginal epithelial cells and Lactobacillus or L. lactis were mixed and incubated at 37° C. for 2 h. After incubation, the cells were then washed five times with PBS to remove non-adherent bacteria. Following the last centrifugation, the cell pellet was transferred to microscope slides, dried, fixed with methanol and stained with 0.1% crystal violet. Three replicates were made for each sample and 100 randomly chosen cells from each replicate were examined using a light microscope under oil immersion and the results expressed as number of bacteria per cell. VEC not incubated with lactobacilli or L. lactis were included as negative control.

Statistical analyses. Data are presented as mean±SD. Significant differences (P<0.05) between means were identified by one-way Analysis of Variance (ANOVA) followed by Duncan's test procedures using SPSS 20. All experiments were performed in triplicate and repeated three times.

### REFERENCES

1. Turroni, F. et al. Molecular dialogue between the human gut microbiota and the host: a Lactobacillus and Bifidobacterium perspective. Cell. Mol. Life Sci. 71, 183-203 (2014).
2. Cano-Garrido, O., Seras-Franzoso, J. & Garcia-Fruitós, E. Lactic acid bacteria: reviewing the potential of a promising delivery live vector for biomedical purposes. Microb. Cell Fact., 14, 137 (2015).
3. Fukiya, S., Sakanaka, M. & Yokota, A. genetic manipulation and gene modification technologies in bifidobacteria. The Bifidobacteria and Related Organisms: Biology, Taxonomy, Applications. Chapter 15, 243-259 (2018).
4. Biswas, I., Gruss, A., Ehrlich, S. D. & Maguin, E. High-efficiency gene inactivation and replacement system for gram-positive bacteria. J. Bacteriol. 175, 3628-3635 (1993).
5. Canosa, I., Lurz, R., Rojo, F. & Alonso, J. C. β Recombinase Catalyzes Inversion and Resolution between Two Inversely Oriented six Sites on a Supercoiled DNA Substrate and Only Inversion on Relaxed or Linear Substrates. J. Biol. Chem. 273, 13886-13891 (1998).
6. Marcotte, H. et al. Characterization and complete genome sequences of L. rhamnosus DSM 14870 and L. gasseri DSM 14869 contained in the EcoVag® probiotic vaginal capsules. Microbiol. Res. 205, 88-98(2017).
7. Goh, Y. J. et al. Development and Application of a upp-Based Counterselective Gene Replacement System for the Study of the S-Layer Protein SlpX of Lactobacillus acidophilus NCFM. Appl. Environ. Microbiol. 75, 3093-3105 (2009).
8. Lim, B., Zimmermann, M., Barry, N. A. &Goodman, A. L. Engineered regulatory systems modulate gene expression of human commensals in the gut. Cell 169, 547-558e15 (2017).
9. Kruger, C. et al. In situ delivery of passive immunity by lactobacilli producing singe-chain antibodies. Nat. Biotechnol. 20, 702-706 (2002).
10. Okano, K. et al. Metabolic engineering of Lactobacillus plantarum for direct L-lactic acid production from raw corn starch. Biotechnol. J. 13, e1700517 (2018).
11. Hidalgo-Cantabrana, C. et al. A single mutation in the gene responsible for the mucoid phenotype of Bifidobacterium animalis subsp. lactis confers surface and functional characteristics. Appl. Environ. Microbiol. 81, 7960-7968 (2015).
12. Kimmel, S. A., Roberts, R. F., & Ziegler, G. R. Optimization of Exopolysaccharide Production by Lactobacillus delbrueckii subsp. bulgaricus RR Grown in a Semidefined Medium. Appl. Environ. Microbiol. 64, 659-664 (1998).
13. De Keersmaecker, S. C. J. et al. Flow Cytometric Testing of Green Fluorescent Protein-Tagged Lactobacillus rhamnosus GG for Response to Defensins. Appl. Environ. Microbiol. 72, 4932-4930 (2006).
Alvarez B, Krogh-Andersen K, Tellgren-Roth C, Martinez N, Gunaydin G, Lin Y, Martin M C, Alvarez M A, Hammarstrom L, Marcotte H. 2015. An EPS-deficient mutant of Lactobacillus rhamnosus GG efficiently displays a protective llama antibody fragment against rotavirus on its surface. Appl Environ Microbiol 81:5784-5793.
Azcarate-Peril M A, Altermann E, Goh Y J, Tallon R, Sanozky-Dawes R B, Pfeiler E A, O'Flaherty S, Buck B L, Dobson A, Duong T, Miller M J, Barrangou R, Klaenhammer T R. 2008. Analysis of the genome sequence of Lactobacillus gasseri ATCC 33323 reveals the molecular basis of an autochthonous intestinal organism. Appl Environ Microbiol 74:4610-4625.
Bae T, Schneewind O. 2003. The YSIRK-G/S motif of staphylococcal protein A and its role in efficiency of signal peptide processing. J Bacteriol 185:2910-2919.
Boekhorst J, De Been M W H J, Kleerebezem M, Siezen R J. 2005. Genome-wide detection and analysis of cell wall-bound proteins with LPxTG-like sorting motifs. J Bacteriol 187:4928-4934.
Bolton M, van der Straten A, Cohen C R. 2008. Probiotics: potential to prevent HIV and sexually transmitted infections in women. Sex Transm Dis 35:214-225.
Buck B L, Altermann E, Svingerud T, Klaenhammer T R. 2005. Functional analysis of putative adhesion factors in Lactobacillus acidophilus NCFM. Appl Environ Microbiol 71:8344-8351.
Call E K, Klaenhammer T R. 2013. Relevance and application of sortase and sortase-dependent proteins in lactic acid bacteria. Front Microbiol 4:73.
Danielsson D, Teigen P K, Moi H. 2011. The genital econiche: focus on microbiota and bacterial vaginosis. Ann N Y Acad Sci 1230:48-58.
Denou E, Pridmore R D, Berger B, Panoff J M, Arigoni F, Brüssow H. 2008. Identification of genes associated with the long-gut-persistence phenotype of the probiotic Lactobacillus johnsonii strain NCC533 using a combination of genomics and transcriptome analysis. J Bacteriol 190:3161-3168.
Dertli E, Mayer M J, Narbad A. 2015. Impact of the exopolysaccharide layer on biofilms, adhesion and resistance to stress in Lactobacillus johnsonii F19785. BMC Microbiol 15:8.
de Ruyter P G, Kuipers O P, de Vos W M. 1996. Controlled gene expression systems for Lactococcus lactis with the food-grade inducer nisin. Appl Environ Microbiol 62:3662-3667.
Edelman S M, Lehti T A, Kainulainen V, Antikainen J, Kylvä jä R, Baumann M, Westerlund-Wikström B, Korhonen T K. 2012. Identification of a high-molecular-mass Lactobacillus epithelium adhesin (LEA) of Lactobacillus crispatus ST1 that binds to stratified squamous epithelium. Microbiology 158:1713-1722.
Etzold S, Kober O I, Mackenzie D A, Tailford L E, Gunning A P, Walshaw J, Hemmings A M, Juge N. 2014. Structural basis for adaptation of lactobacilli to gastrointestinal mucus. Environ Microbiol 16:888-903.
Horn N, Wegmann U, Dertli E, Mulholland F, Collins S R A, Waldron K W, Bongaerts R J, Mayer M J, Narbad A. 2013. Spontaneous mutation reveals influence of exopolysaccharide on Lactobacillus johnsonii surface characteristics. PLoS ONE 8:e59957.
Jensen H, Roos S, Jonsson H, Rud 1, Grimmer S, Van Pijkeren J P, Britton R A, Axelsson L. 2014. Role of Lactobacillus reuteri cell and mucus-binding protein A (CmbA) in adhesion to intestinal epithelial cells and mucus in vitro. Microbiology 160:671-681.
Jolly L, Stingele F. 2001. Molecular organization and functionality of exopolysaccharide gene clusters in lactic acid bacteria. Int Dairy J 11:733-745.
Jones S E, Versalovic J. 2009. Probiotic Lactobacillus reuteri biofilms produce antimicrobial and anti-inflammatory factors. BMC Microbiol 9:35.
Juge N. 2012. Microbial adhesins to gastrointestinal mucus. Trends Microbiol 20:30-39.
Kankainen M, Paulin L, Tynkkynen S, von Ossowski I, Reunanen J, Partanen P, Satokari R, Vesterlund S, Hendrickx A P A, Lebeer S, De Keersmaecker S C J, Vanderleyden J, Hämäläinen T, Laukkanen S, Salovuori N, Ritari J, Alatalo E, Korpela R, Mattila-Sandholm T, Lassig A, Hatakka K, Kinnunen K T, Karjalainen H, Saxelin M, Laakso K, Surakka A, Palva A, Salusjärvi T, Auvinen P, de Vos W M. 2009. Comparative genomic analysis of Lactobacillus rhamnosus GG reveals pili containing a human-mucus binding protein. Proc Natl Acad Sci USA 106:17193-17198.
De Keersmaecker S C J, Braeken K, Verhoeven T L A, Ve'lez M P, Lebeer S, Vanderleyden J, Hols P. 2006. Flow cytometric testing of green fluorescent protein-tagged Lactobacillus rhamnosus GG for response to defensins. Appl Environ Microbiol 72:4923-4930.
Kuipers O P, de Ruyter P G, Kleerebezem M, de Vos W M. 1998. Quorum sensing-controlled gene expression in lactic acid bacteria. J Biotechnol 64:15-21.
Larsson P G, Brandsborg E, Forsum U, Pendharkar S, Andersen K K, Nasic S, Hammarstrom L, Marcotte H. 2011. Extended antimicrobial treatment of bacterial vaginosis combined with human lactobacilli to find the best treatment and minimize the risk of relapses. BMC Infect Dis 11:223.
Larsson P G, Stray-Pedersen B, Ryttig K R, Larsen S. 2008. Human lactobacilli as supplementation of clindamycin to patients with bacterial vaginosis reduce the recurrence rate; a 6-month, double-blind, randomized, placebo-controlled study. BMC Womens Health 8:3.
Lebeer S, Claes I, Tytgat H L, Verhoeven T L, Marien E, von Ossowski I, Reunanen J, Palva A, Vos W M, Keersmaecker S C, Vanderleyden J. 2012. Functional analysis of Lactobacillus rhamnosus GG pili in relation to adhesion and immunomodulatory interactions with intestinal epithelial cells. Appl Environ Microbiol 78:185-193.
Lebeer S, Vanderleyden J, De Keersmaecker S C J. 2008. Genes and molecules of lactobacilli supporting probiotic action. Microbiol Mol Biol Rev 72:728-764.
Lebeer S, Vanderleyden J, De Keersmaecker S C J. 2010. Host interactions of probiotic bacterial surface molecules: comparison with commensals and pathogens. Nat Rev Microbiol 8:171-184.
Lebeer S, Verhoeven T L A, Francius G, Schoofs G, Lambrichts I, Dufrene Y, Vanderleyden J, De Keersmaecker S C J. 2009. Identification of a gene cluster for the biosynthesis of a long, galactose-rich exopolysaccharide in Lactobacillus rhamnosus GG and functional analysis of the priming glycosyltransferase. Appl Environ Microbiol 75:3554-3563.
Lebeer S, Verhoeven T L A, Ve'lez M P, Vanderleyden J, De Keersmaecker S C J. 2007. Impact of environmental and genetic factors on biofilm formation by the probiotic strain Lactobacillus rhamnosus GG. Appl Eviron Microb 73:6768-6775.
Leccese Terraf M C, Mendozal L M, Juárez Tomas M S, Silva C, Nader-Macias M E F. 2014. Phenotypic surface properties (aggregation, adhesion and biofilm formation) and presence of related genes in beneficial vaginal lactobacilli. J Appl Microbiol 117:1761-1772.
Lee I-C, Caggianiello G, van Swam I I, Taverne N, Meijerink M, Bron P A, Spano G, Kleerebezema M. 2016. Strain-specific features of extracellular polysaccharides and their impact on Lactobacillus plantarum-host interactions. Appl Eviron Microbiol 82:3959-3970.
MacKenzie D A, Tailford L E, Hemmings A M, Juge N. 2009. Crystal structure of a mucus-binding protein repeat reveals an unexpected functional immunoglobulin binding activity. J Biol Chem 284:32444-32453.
Malik S, Petrova M I, Claes I J J, Verhoeven T L A, Busschaert P, Vaneechoutte M, Lievens B, Lambrichts I, Siezen R J, Balzarini J, Vanderleyden J, Lebeer S. 2013. The high auto-aggregative and adhesive phenotype of the vaginal Lactobacillus plantarum strain CMPG5300 is sortase-dependent. Appl Environ Microbiol 79:4576-4585.
Malik S, Petrova M I, Imholz N C E, Verhoeven T L A, Noppen S, Van Damme E J M, Liekens S, Balzarini J, Schols D, Vanderleyden J, Lebeer S. 2016. High mannose-specific lectin Msl mediates key interactions of the vaginal Lactobacillus plantarum isolate CMPG5300. Sci Rep 6:37339.
Marcotte H, Andersen K K, Lin Y, Zuo F L, Zeng Z, Larsson P G, Brandsborg E, Brønstad G, Hammarström L. 2017. Characterization and complete genome sequences of L. rhamnosus DSM 14870 and L. gasseri DSM 14869 contained in the EcoVag® probiotic vaginal capsules. Microbiol Res 205:88-98.
Marraffini L A, Dedent A C, Schneewind 0.2006. Sortases and the art of anchoring proteins to the envelopes of gram-positive bacteria. Microbiol Mol Biol Rev 70:192-221.
Michel J L, Madoff L C, Olson K, Kling D E, Kasper D L, Ausubel F M. 1992. Large, identical, tandem repeating units in the C protein alpha antigen gene, bca, of group B streptococci. Proc Natl Acad Sci USA 89:10060-10064.
Mora D, Maguin E, Masiero M, Parini C, Ricci G, Manachini P L, Daffonchio D. 2004. Characterization of urease genes cluster of Streptococcus thermophilus. J Appl Microbiol 96:209-219.
von Ossowski I, Satokari R, Reunanen J, Lebeer S, De Keersmaecker S C, Vanderleyden J, de Vos W M, Palva A. 2011. Functional characterization of a mucus-specific LPXTG surface adhesin from probiotic Lactobacillus rhamnosus GG. Appl Environ Microbiol 77:4465-4472.
Parolin C, Marangoni A, Laghi L, Foschi C, Nahui Palomino R A, Calonghi N, Cevenini R, Vitali B. 2015. Isolation of vaginal lactobacilli and characterization of Anti-Candida activity, PLOS ONE 10:e0131220.
Pendharkar S, Brandsborg E, Hammarstrom L, Marcotte H, Larsson P G. 2015. Vaginal colonization by probiotic lactobacilli and clinical outcome in women conventionally treated for bacterial vaginosis and yeast infection. BMC Infect Dis 15:255.
Pendharkar S, Magopane T, Larsson P G, de Bruyn G, Gray G E, Hammarstrom L, Marcotte H. 2013. Identification and characterisation of vaginal lactobacilli from South African women. BMC Infect Dis 13:43.
Polak-Berecka M, Wako A, Paduch R, Skrzypek T, Sroka-Bartnicka A. 2014. The effect of cell surface components on adhesion ability of Lactobacillus rhamnosus. Antonie Van Leeuwenhoek 106:751-762.
Ravel J, Gajer P, Abdo Z, Schneider G M, Koenig S S, McCulle S L, Karlebach S, Gorle R, Russell J, Tacket C O, Brotman R M, Davis C C, Ault K, Peralta L, Forney L J. 2011. Vaginal microbiome of reproductive-age women. Proc Natl Acad Sci USA 108 Suppl 1:4680-4687.
Reid G, Dols J, Miller W. 2009. Targeting the vaginal microbiota with probiotics as a means to counteract infections. Curr Opin Clin Nutr Metab Care 12:583-587.
Ronnqvist P D, Forsgren-Brusk U B, Grahn-Hakansson E E. 2006. Lactobacilli in the female genital tract in relation to other genital microbes and vaginal pH. Acta Obstet Gynecol Scand 85:726-735.
Rojas M, Ascencio F, Conway P L. 2002. Purification and characterization of a surface protein from Lactobacillus fermentum 104R that binds to porcine small intestinal mucus and gastric mucin. Appl Environ Microbiol 68:2330-2336.
Ruas-Madiedo P, Hugenholtz J, Zoon P. 2002. An overview of the functionality of exopolysaccharides produced by lactic acid bacteria. Int Dairy J 12:163-171.
Sambrook J, Fritsch E F, Maniatis T. 1989. Molecular cloning: a laboratory manual. Cold Spring Harbor Laboratory Press, Cold Spring Harbor, N.Y.
Stalhammar-Carlemalm M, Areschoug T, Larsson C, Lindahl G. 1999. The R28 protein of Streptococcus pyogenes is related to several group B streptococcal surface proteins confers protective immunity and promotes binding to human epithelial cells. Mol Microbiol 33:208-219.
Schmittgen T D, Livak K J. 2008. Analyzing real-time PCR data by the comparative CT method. Nat Protoc 3:1101-1108.
Turner M S, Hafner L M, Walsh T, Giffard P M. 2003. Peptide surface display and secretion using two LPXTG-containing surface proteins from Lactobacillus fermentum BR11. Appl Environ Microbiol 69:5855-5863.
Vasquez A, Jakobsson T, Ahrne S, Forsum U, Molin G. 2002. Vaginal Lactobacillus flora of healthy Swedish women. J Clin Microbiol 40:2746-2749.
Walter J, Chagnaud P, Tannock G W, Loach D M, Dal Bello F, Jenkinson H F, Hammes W P, Hertel C. 2005. A highmolecular-mass surface protein (Lsp) and methionine sulfoxide reductase B (MsrB) contribute to the ecological performance of Lactobacillus reuteri in the murine gut. Appl Environ Microbiol 71:979-986.
Wästfelt M, Stâlhammar-Carlemalm M, Delisse A-M, Cabezon T, Lindahl G. 1996. Identification of a family of streptococcal surface proteins with extremely repetitive structure. J Biol Chem 271:18892-18897.
Younes J A, van der Mei H C, van den Heuvel E, Busscher H J, Reid G. 2012. Adhesion forces and coaggregation between vaginal staphylococci and lactobacilli. PLOS ONE 7:e36917.
Živković M, Miljkovie M S, Ruas-Madiedo P, Markelie M B, Veljović K, Tolinaeki M, Soković S, Korać A, Golić N. 2016. EPS-SJ exopolisaccharide produced by the strain Lactobacillus paracasei subsp. paracasei BGSJ2-8 is involved in adhesion to epithelial intestinal cells and decrease on E. coli association to Caco-2 cells. Front Microbiol 7:28.

1. A circular DNA vector comprising:
(a) a selectable marker gene sequence, wherein said selectable marker gene sequence is operably linked to a first promoter sequence,
(b) a multiple cloning site, wherein said multiple cloning site optionally comprises a gene targeting sequence,
(c) a sequence encoding a site-specific recombinase, wherein said sequence encoding said site-specific recombinase is operably linked to a second promoter sequence, wherein said second promoter is inducible,
(d) a replicon sequence, and
(e) two target sites for said site-specific recombinase,
wherein said vector further comprises a first region flanked on each side by one of said target sites for said site-specific recombinase and wherein said first region comprises (a) and (b) with the proviso that (c) and (d) are not within said first region.

2-54. (canceled)

55. The circular DNA vector of claim 1, wherein said selectable marker is an antibiotic resistance gene selected from the group consisting of a chloramphenicol resistance gene, spectinomycin resistance gene, tetracycline resistance gene and erythromycin resistance gene.

56. The circular DNA vector according to claim 1, wherein said site-specific recombinase is a site-specific serine recombinase.

57. The circular DNA vector according to claim 1, wherein said site-specific recombinase is selected from the group consisting of beta-recombinase, Cre-recombinase, FLP-recombinase and PhiC31 integrase.

58. The circular DNA vector according to claim 1, wherein said replicon sequence is a prokaryotic replicon sequence.

59. The circular DNA vector according to claim 1, wherein said replicon sequence encodes a origin of replication (ori) and replication initiator protein (Rep protein).

60. The circular DNA vector according to claim 1, wherein said replicon sequence is a replicon sequence permissive for replication of said vector in a prokaryote host cell.

61. The circular DNA vector according to claim 1, wherein said replicon sequence is a replicon sequence permissive for replication of said vector in Lactobacilli or Bifidobacteria.

62. The circular DNA vector according to claim 1, wherein said replicon sequence is a replicon sequence that allows replication of said vector in E. coli.

63. The circular DNA vector according to claim 1, wherein said replicon sequence is a replicon sequence permissive for replication of said vector in E. coli and an additional prokaryotic host cell.

64. The circular DNA vector according to claim 1, wherein said replicon sequence is a replicon sequence permissive for replication of said vector in a least one host cell selected from the group consisting of a Lactobacillus gasseri, Lactobacillus rhamnosus, Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

65. The circular DNA vector according to claim 1, wherein said second promoter sequence is an inducible prokaryotic promoter selected from the group consisting of a sakacin-inducible promoter, a tetracycline-inducible promoter D-xylose-inducible promoter, lactose-inducible promoter, IPTG-inducible promoter, nisin inducible promoter, a bile inducible promoter, a bacteriocin-inducible promoter and a synthetic inducible promoter.

66. The circular DNA vector according to claim 1, wherein said two target sites for said site-specific recombinase are orientated such that the product of site-specific recombination between two target sites for said site-specific recombinase is a first circular DNA product comprising (a) and (b) and a second circular DNA product comprising (c) and (d).

67. The circular DNA vector according to claim 1, wherein said vector further comprises a gene targeting sequence inserted in the multiple cloning site.

68. A method for introducing recombination between a circular DNA vector and a target region of the genome of a host cell, said method comprising:
(i) introducing a circular DNA vector comprising a gene targeting sequence according to claim 67 in a host cell, wherein said gene targeting sequence comprises flanking sequences comprising at least about 200 consecutive nucleotides having a sequence identity of at least 80% to the corresponding region of the target region of the host cell genome,
(ii) inducing expression of the site-specific recombinase encoded by said circular DNA vector and, allowing site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the gene targeting sequence and the target region of the genome of said host cell, and
(iv) selecting a host cell, wherein (a) have been excised from the genome of the host cell obtained under (iii) by a second homologous recombination event between the flanking sequences of the gene targeting sequence and the target region of the genome.

69. A method for generating and selecting a host cell having a mutation in a target gene of the genome of a host cell, said method comprising:
(i) introducing a circular DNA vector comprising a gene targeting sequence according to claim 67 in a host cell, wherein said gene targeting sequence comprises flanking sequences comprising at least about 200 consecutive nucleotides having a sequence identity of at least 80% to the corresponding region of the target gene of the host cell genome,
(ii) inducing expression of the site-specific recombinase encoded by said circular DNA vector and, allow site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the gene targeting sequence and the target region of the genome of said host cell, and
(iv) selecting a host cell, wherein (a) have been excised from the genome of the host cell obtained under (iii) by a second homologous recombination event between the flanking sequences of the gene targeting sequence and the target region of the genome and, wherein said host cell comprises a mutation in said target region.

70. A method for generating a host cell having loss of function in a target gene of the genome of a host cell, said method comprising:
(i) introducing a circular DNA vector comprising a gene targeting sequence according to claim 67 in a host cell, wherein said gene targeting sequence comprises flanking sequences comprising at least about 200 consecutive nucleotides having a sequence identity of at least 80% to the corresponding region of the target gene of the host cell genome,
(ii) inducing expression of the site-specific recombinase encoded by said circular DNA vector and, allowing site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the gene targeting sequence and the target region of the genome of said host cell, and
(iv) selecting a host cell, wherein (a) have been excised from the genome of the host cell obtained under (iii) by a second homologous recombination event between the flanking sequences of the gene targeting sequence and the target region of the genome and, wherein said host cell comprises a loss of function of said target gene.

71. A method for generating a host cell having gain of function in a target gene of the genome of a host cell, said method comprising:
(i) introducing a circular DNA vector comprising a gene targeting sequence according to claim 67 in a host cell, wherein said gene targeting sequence comprises flanking sequences comprising at least about 200 consecutive nucleosides having a sequence identity of at least 80% to the target gene of the host cell genome,
(ii) inducing expression of the site-specific recombinase encoded by said circular DNA vector and, allowing site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the gene targeting sequence and the target region of the genome of said host cell, and
(iv) selecting a host cell, wherein (a) have been excised from the genome of the host cell obtained under (iii) by a second homologous recombination event between the flanking sequences of the gene targeting sequence and the target region of the genome and, wherein said host cell comprises a gain of function of said target gene.

72. A method for preparing a host cell expressing a recombinant polypeptide, said method comprising:
(i) introducing a circular DNA vector comprising a gene targeting sequence according to claim 67 in a host cell, wherein said gene targeting sequence comprises flanking sequences comprising at least about 200 consecutive nucleosides having a sequence identity of at least 80% to the corresponding region of the target region of the host cell genome, and wherein said gene targeting sequence encodes a recombinant polypeptide,
(ii) inducing expression of the site-specific recombinase encoded by said circular DNA vector and, allowing site-specific recombination between the target sites of said site-specific recombinase to produce a first circular DNA product comprising (a) and (b) and a second circular DNA product comprising (c) and (d),
(iii) selecting a host cell, wherein said first circular DNA product comprising (a) and (b) is integrated at the target region of the genome by a first single-crossover homologous recombination event between the flanking sequences of the gene targeting sequence and the target region of the genome of said host cell, and
(iv) selecting a host cell, wherein (a) have been excised from the genome of the host cell obtained under (iii) by a second homologous recombination event between the flanking sequences of the gene targeting sequence and the target region of the genome.

73. The method according to claim 72, wherein said recombinant polypeptide is a polypeptide selected from the group consisting of monoclonal antibodies, humanized monoclonal antibodies, chimeric antibodies, single-domain antibodies, camelid antibodies, enzymes, cytokines, hormones and blood-clotting proteins.

74. The method according to claim 68, wherein the flanking sequences comprise consecutive nucleotides in the range of 200 to 1500 consecutive nucleosides.

75. The method according to claim 68, wherein the flanking sequences have a sequence identity of at least 85% to the corresponding region of the target gene of the host cell genome.

76. The method according to claim 68, wherein said selection under (iii) uses the selectable marker gene sequence (a) of the circular DNA vector.

77. The method according to claim 68, wherein said selection under (iii) uses PCR and/or DNA sequencing.

78. The method according to claim 68, wherein said host cell selected under (iv) is selected by counterselection.

79. The method according to claim 68, wherein said host cell selected under (iv) is negative for said selectable marker.

80. The method according to claim 68, wherein said host cell selected under (iv) is selected using PCR.

81. The method according to claim 68, wherein the product of (iv) is a host cell comprising a deletion of the target region, a partial deletion of the target region, a sequence insertion at the target region, a point mutation of the target region, or a sequence replacement of the target region.

82. The method according to claim 68, wherein said host cell is a prokaryote.

83. The method according to claim 68, wherein said host cell a Lactobacilli or a Bifidobacteria.

84. The method according to claim 68, wherein said host cell is selected from the group consisting of a Lactobacillus gasseri, Lactobacillus rhamnosus, Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

85. The method according to claim 68, wherein said replicon comprises a replication origin for E. coli.

86. The method according to claim 68, wherein said replicon comprises repA encoding Regulatory protein RepA or repB encoding RepFIB replication protein A or RepC encoding Replication initiation protein.

87. The method according to claim 68, wherein said circular DNA vector is introduced by transformation.

88. The method according to claim 68, wherein said target gene encodes a cell surface protein.

89. The method according to claim 68, wherein said cell surface protein is a sortase dependent protein (SDP) or a S-layer protein.

90. The method according to claim 68, wherein said target gene encodes a cell protein involved in the biosynthesis of a cell surface molecule.

91. The method according to claim 68, wherein said cell surface molecule is a exopolysaccharide (EPS).

92. The method according to any one claim 68, wherein said target gene encodes a protein involved in bacterial adherence, auto-aggregation and/or biofilm formation.

93. The method according to claim 68, wherein said target gene is selected from the group consisting of N506_1709, N506_1778, N506_0396, N506_0397, N506_0398, N506_0399, N506_0400, N506_0401, N506_0402, N506_0403, N506_0404, N506_0405, N506_0406, N506_0407, N506_0408, N506_0409, N506_0410 and N506_0411.

94. The method according to claim 68, wherein said host cell is a Lactobacillus gasseri.

95. A recombinant host cell obtained by the method of claim 68.

96. A method of introducing a gene sequence in the genome of a host cell comprising introducing the circular DNA vector of claim 1 in a host cell.

97. The method of claim 96, further comprising selecting said host cell for having an increased tissue adhesion.

98. The method according to claim 97, wherein said host cell is a bacterium selected for having an increased tissue adhesion to human vaginal tissue.

99. The method according to claim 98, wherein said host cell a Lactobacilli or Bifidobacteria bacterium.

100. The method according to claim 99, wherein said host cell is selected from the group consisting of a Lactobacillus gasseri, Lactobacillus rhamnosus, Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

101. The method according to claim 96, wherein the circular vector introduces a deletion of a target region, partial deletion of a target region, a sequence insertion at a target region, a point mutation of a target region, or a sequence replacement of a target region.

102. The method according to claim 96, wherein said gene sequence is expressed in said host cell.

103. The method according to claim 96, wherein said gene sequence blocks expression of an endogenous host gene.

104. The method according to claim 96, wherein said gene sequence replaces a corresponding endogenous host gene.

105. The method according to claim 96, wherein said host cell is a Lactobacilli or a Bifidobacteria.

106. The method according to claim 96, wherein said host cell is selected from the group consisting of a Lactobacillus gasseri, Lactobacillus rhamnosus, Lactobacillus paracasei, Lactobacillus plantarum, Lactobacillus acidophilus, Lactobacillus fermentum, Lactobacillus crispatus, Lactobacillus jensenii, Lactobacillus vaginalis, Lactobacillus iners, Lactobacillus reuteri, Lactobacillus casei, Lactobacillus bulgaricus, Lactobacillus curvatus, Lactobacillus delbrueckii and Lactobacillus johnsonii.

107. The method according to claim 106, wherein said target gene is selected from the group consisting of N506_1709, N506_1778, N506_0396, N506_0397, N506_0398, N506_0399, N506_0400, N506_0401, N506_0402, N506_0403, N506_0404, N506_0405, N506_0406, N506_0407, N506_0408, N506_0409, N506_0410 and N506_0411.

108. The method according to claim 107, wherein said host cell is a Lactobacillus gasseri.


SEQUENCE LISTING (58 sequences)

SEQ ID NO: 1 - DNA, 29 bp, Artificial Sequence
  SIX-F1
gaccggtcgacaattattagggggagaag

SEQ ID NO: 2 - DNA, 29 bp, Artificial Sequence
  SIX-R1
cgccagtcgacgagtcgtgcataaccaat

SEQ ID NO: 3 - DNA, 29 bp, Artificial Sequence
  SIX-F2
gaccgctgcagaattattagggggagaag

SEQ ID NO: 4 - DNA, 29 bp, Artificial Sequence
  SIX-R2
cgccaaagcttgagtcgtgcataaccaat

SEQ ID NO: 5 - DNA, 53 bp, Artificial Sequence
  linker-F
gatctgagctcatgcatgggcccgatcgctagcggccgcatgcggatcctgca

SEQ ID NO: 6 - DNA, 45 bp, Artificial Sequence
  Linker-R
ggatccgcatgcggccgctagcgatcgggcccatgcatgagctca

SEQ ID NO: 7 - DNA, 31 bp, Artificial Sequence
  upp-up-F
taattgggcccaaataatggaaactaagatt

SEQ ID NO: 8 - DNA, 45 bp, Artificial Sequence
  upp-up-R
gttcagcatgctaacaagagctcagataaatgtttcttaaatcgt

SEQ ID NO: 9 - DNA, 45 bp, Artificial Sequence
  upp-down-F
caggagagctcttgttcggatccaagtaattttactcaaaaatct

SEQ ID NO: 10 - DNA, 33 bp, Artificial Sequence
  upp-down-R
ttacagcatgcaaaacgcaaattacaggaagag

SEQ ID NO: 11 - DNA, 23 bp, Artificial Sequence
  uppleft-F
ttaccagattttgaaattgagtt

SEQ ID NO: 12 - DNA, 23 bp, Artificial Sequence
  uppright-R
agtaaagcgtatctcctaactct

SEQ ID NO: 13 - DNA, 25 bp, Artificial Sequence
  plrec-R
agatttattgagaggagggattatt

SEQ ID NO: 14 - DNA, 23 bp, Artificial Sequence
  plrec-R
cgtttgttgaactaatgggtgct

SEQ ID NO: 15 - DNA, 24 bp, Artificial Sequence
  plrecSC-F
aaagttttcgggctactctctcct

SEQ ID NO: 16 - DNA, 26 bp, Artificial Sequence
  plrecSC-R
ggaattgtcagataggcctaatgact

SEQ ID NO: 17 - DNA, 23 bp, Artificial Sequence
  uppseq-F
gaacaattagtcctgcttatatg

SEQ ID NO: 18 - DNA, 23 bp, Artificial Sequence
  uppseq-R
cgactacagatttctcattcact

SEQ ID NO: 19 - DNA, 30 bp, Artificial Sequence
  400upstream-F
acacagagctcagctggtgaaatggatggc

SEQ ID NO: 20 - DNA, 37 bp, Artificial Sequence
  400upstream-R
ttaacttttctccttaaaataaccaaaatcttttctt

SEQ ID NO: 21 - DNA, 33 bp, Artificial Sequence
  400downstream-F
ttggttattttaaggagaaaagttaaaagtatg

SEQ ID NO: 22 - DNA, 31 bp, Artificial Sequence
  400downstream-R
accaagctagctgtatcacttttggtattga

SEQ ID NO: 23 - DNA, 22 bp, Artificial Sequence
  Seq1-F
attgggttctaacagaatgcgt

SEQ ID NO: 24 - DNA, 23 bp, Artificial Sequence
  INTZ-R
ttctccccctaataattctgcag

SEQ ID NO: 25 - DNA, 23 bp, Artificial Sequence
  INTZ-F
ggtttttatattacagctccaag

SEQ ID NO: 26 - DNA, 22 bp, Artificial Sequence
  Seq1-R
aatcctgaatcactttcggttt

SEQ ID NO: 27 - DNA, 30 bp, Artificial Sequence
  1778upstream-F
caacggagctccgaaactaatggcatcaat

SEQ ID NO: 28 - DNA, 43 bp, Artificial Sequence
  1778upstream-R
gtttttggtgcagagaaatttatatataaaataacgattttgt

SEQ ID NO: 29 - DNA, 47 bp, Artificial Sequence
  1778downstream-F
ttgtataggagaaacaaggtttcttgataataaataacattaatagc

SEQ ID NO: 30 - DNA, 35 bp, Artificial Sequence
  1778downstream-R
ctttaaggatccaaaagaagaagctagaaaggctt

SEQ ID NO: 31 - DNA, 24 bp, Artificial Sequence
  Seq2-F
cacataataccagcagtcaacgaa

SEQ ID NO: 32 - DNA, 25 bp, Artificial Sequence
  Seq2-R
gtgctccaagtagtatcatagcgat

SEQ ID NO: 33 - DNA, 34 bp, Artificial Sequence
  1709upstream-F
ttcctggagctcaaactttattttgttctgccaa

SEQ ID NO: 34 - DNA, 43 bp, Artificial Sequence
  1709upstream-R
atgttatttattatcaagaaaccttgtttctcctatacaatgt

SEQ ID NO: 35 - DNA, 47 bp, Artificial Sequence; identical to SEQ ID NO: 29
  1709downstream-F

SEQ ID NO: 36 - DNA, 35 bp, Artificial Sequence; identical to SEQ ID NO: 30
  1709downstream-R

SEQ ID NO: 37 - DNA, 24 bp, Artificial Sequence
  Seq3-F
ggctaaaaacagactccatcaatc

SEQ ID NO: 38 - DNA, 25 bp, Artificial Sequence
  Seq3-R
agcccgttctttcttataactttaa

SEQ ID NO: 39 - DNA, 36 bp, Artificial Sequence
  EPS-Promoter-F
aattaggtaccacactgtaaaaataaataagatcct

SEQ ID NO: 40 - DNA, 40 bp, Artificial Sequence
  EPS-Promoter-R
ttaacctcttgtgccatcattttattcctcttttattttt

SEQ ID NO: 41 - DNA, 40 bp, Artificial Sequence
  N506_0400-F
aaaaataaaagaggaataaaatgatggcacaagaggttaa

SEQ ID NO: 42 - DNA, 33 bp, Artificial Sequence
  N506_0400-R
cttttaagcttaatacgcactatttggatgaat

SEQ ID NO: 43 - DNA, 36 bp, Artificial Sequence
  N506_1778-F
tacatcggtaccaaaatatgcggtatgtatttatcg

SEQ ID NO: 44 - DNA, 38 bp, Artificial Sequence
  N506_1778-R
ggtgcaggatccttaattctttttctttcgtttaagtt

SEQ ID NO: 45 - DNA, 38 bp, Artificial Sequence
  N506_1709-F1
aagacagatctcgtaattaaattgatcaagtacattat

SEQ ID NO: 46 - DNA, 23 bp, Artificial Sequence
  N506_1709-R1
acaaaggtaccaccagattccat

SEQ ID NO: 47 - DNA, 25 bp, Artificial Sequence
  N506_1709-F2
ctggtggtacctttgtttcaaaagt

SEQ ID NO: 48 - DNA, 39 bp, Artificial Sequence
  N506_1709-R2
attatgagctcttatctaattcggtgttttcttctactt

SEQ ID NO: 49 - DNA, 37 bp, Artificial Sequence
  N506_1709Re-F
gagaaaccatggatgctatctaaaaataattttcatg

SEQ ID NO: 50 - DNA, 51 bp, Artificial Sequence
  N506_1709 Re-R
ttaatgagctcttacgcttccggttctctaattcggtgttttcttctactt

SEQ ID NO: 51 - DNA, 23 bp, Artificial Sequence
  L. gasseri 16S-F
acccttgtcattagttgccatca

SEQ ID NO: 52 - DNA, 22 bp, Artificial Sequence
  L. gasseri 16S-R
gcttctcgttgtaccgtccatt

SEQ ID NO: 53 - DNA, 26 bp, Artificial Sequence
  1778-F
agaccttagagagcaagtcattatcg

SEQ ID NO: 54 - DNA, 24 bp, Artificial Sequence
  1778-R
ttggttattaggaagttcgtcgtt

SEQ ID NO: 55 - DNA, 21 bp, Artificial Sequence
  1709-F
attggaacgatttgaagagcg

SEQ ID NO: 56 - DNA, 23 bp, Artificial Sequence
  1709-R
gaatcagtagtgtgggaaccgac

SEQ ID NO: 57 - DNA, 21 bp, Artificial Sequence
  L. lactis 16S-F
tcgtgtcgtgagatgttgggt

SEQ ID NO: 58 - DNA, 23 bp, Artificial Sequence
  L. lactis 16S-R
gtcataaggggcatgatgatttg